(19) United States
(12) Patent Application Publication
(10) Pub. No.: US 2026/0294939 A1
(43) Pub. Date: Oct. 1, 2026

(54) PSILOCYBIN COMPOSITIONS, METHODS OF MAKING AND METHODS OF USING THE SAME

(71) Applicant: Compass Pathfinder Limited, Altrincham (GB)

(72) Inventors: David Philip ELDER, Hertfordshire (GB); Nigel RICHARDSON, Stevenage (GB)

(73) Assignee: Compass Pathfinder Limited, Altrincham (GB)

(21) Appl. No.: 19/702,612

(22) Filed: Jun. 9, 2026

Related U.S. Application Data
(63) Continuation of application No. 18/285,109, filed on Mar. 30, 2022.
(60) Provisional application No. 63/168,055, filed on Mar. 30, 2021.

(51) Int. Cl. A61K 31/661; A61K 9/20; A61K 9/48

(52) U.S. Cl. CPC A61K 31/661; A61K 9/2059; A61K 9/4833; A61K 9/4866

(57) ABSTRACT

The present disclosure provides pharmaceutical compositions comprising a therapeutically effective amount of psilocybin and one or more pharmaceutically acceptable excipients. Methods of making such compositions and use thereof in treating depressive disorders are also disclosed.

### CROSS-REFERENCE TO RELATED APPLICATIONS

The present application is a Continuation of U.S. patent application Ser. No. 18/285,109, filed Sep. 29, 2023, titled “Psilocybin Compositions, Methods of Making and Methods of Using the Same,” which is a National Stage Entry of International Patent Application No. PCT/EP2022/058483, filed Mar. 30, 2022, which claims priority to and benefit of U.S. Provisional Application No. 63/168,055, filed Mar. 30, 2021, the contents of each are hereby incorporated by reference in their entirety.

### BACKGROUND

Psilocybin belongs to a class of drugs referred to psychedelics (“mind-manifesting” drugs). Specifically, psilocybin is considered a 5-hydroxytryptaminergic (serotonergic) psychedelic, as distinguished from other tryptamines such as dimethyltryptamine (DMT), ergolines such as lysergic acid diethylamide (LSD), and phenethylamines such as mescaline. Psilocybin was first isolated from psilocybe mushrooms and later synthesized in a laboratory.

Provided the potential therapeutic applications, there is a need in the art for pharmaceutical compositions of psilocybin that meet regulatory criteria for approved drug products (e.g., stable during product storage and acceptable content uniformity).

### SUMMARY

Psilocybin is a challenging active pharmaceutical ingredient (API) to formulate because of its physical characteristics and potential chemical instability (e.g., facile hydrolysis to psilocin). Crystalline psilocybin is an acicular, rod-like crystal with a high aspect ratio and, as such, has poor flow properties (particularly hydrate forms), which are undesirable for direct mixing formulation scale-up and commercial manufacturing. Also, during pharmaceutical development (see Example 1), Applicant discovered that solid dosage forms containing certain, commonly-used silica-containing excipients provided non-ideal results upon storage of the drug product under ICH stability testing conditions (e.g., drug product discolouration and a loss of potency without a commensurate increase psilocybin related substance impurities). Finally, given the psychedelic properties of psilocybin, it should be administered as a low dose drug (e.g., 1 mg of psilocybin may be used for some indications). Accordingly, a suitable formulation must allow accommodation of low API loadings, but provide pharmaceutically acceptable content uniformity (as measured by European Pharmacopeia 2.96, or USP <905>).

The present disclosure relates to pharmaceutical compositions containing psilocybin and methods of preparing the same.

In one aspect, the present disclosure provides a pharmaceutical composition comprising: a therapeutically effective amount of psilocybin; and one or more pharmaceutically acceptable excipients, wherein potency of the psilocybin in the composition and the mass balance of psilocybin and related substances in the composition remains at industry and pharmaceutically acceptable levels (e.g., European Pharmacopeia, USP) under industry and pharmaceutically acceptable storage conditions (e.g., European Pharmacopeia, USP) and the psilocybin is provided in pharmaceutically acceptable content uniformity (as measured by European Pharmacopeia 2.96. or USP <905>).

In one aspect, the present disclosure provides a pharmaceutical composition comprising: a therapeutically effective amount of psilocybin; and one or more pharmaceutically acceptable excipients, wherein, after storage of the composition at 40° C. and 75% relative humidity for one month, the potency of the psilocybin in the composition decreases by less than 5% and the mass balance of psilocybin and related substances is greater than 97%.

In one aspect, the present disclosure provides a method of making a pharmaceutical composition comprising: (a) direct mixing psilocybin and one or more pharmaceutically acceptable excipients to provide a blend and (b) filling a capsule with the blend to provide the pharmaceutical composition, wherein the content uniformity of the composition complies with the European Pharmacopeia 2.96, or USP <905>.

### BRIEF DESCRIPTION OF THE DRAWINGS

FIG. 1 is a numbered structural formula of psilocybin.

FIG. 2A is a XRPD diffractogram of Polymorph A

FIG. 2B is a XRPD diffractogram of Polymorph A′

FIG. 2C is a XRPD diffractogram of Polymorph B

FIG. 2D is a XRPD diffractogram of a Hydrate A

FIG. 2E is a XRPD diffractogram of an ethanol solvate

FIG. 2F is a XRPD diffractogram of product obtained during development of the process (top)-compared to the diffractograms Polymorph A′ (middle) and Polymorph B (bottom).

FIG. 3A is a DSC and TGA thermograph of Polymorph A

FIG. 3B is a DSC and TGA thermograph of Polymorph A′ FIG. 3C is a DSC thermograph of Polymorph B

FIG. 3D is a DSC and TGA thermograph of Hydrate A

FIG. 3E is a DSC and TGA thermograph of ethanol solvate

FIG. 4 is a form phase diagram showing the inter-relationship of form in water-based systems.

FIG. 5 is a 1H NMR spectrum of psilocybin.

FIG. 6 is a 13C NMR spectrum of psilocybin.

FIG. 7 is a FT-IR Spectrum of psilocybin.

FIG. 8 is a Mass Spectrum of psilocybin.

FIG. 9 shows a non-limiting example of a process flow diagram for preparing psilocybin capsules of the present disclosure.

FIG. 10 shows the mean assay plot for the control composition from the stability study of Example 4.

FIG. 11 shows the mean assay plot for the low level SMCC composition from the stability study of Example 4.

FIG. 12 shows the mean assay plot for the 1 mg capsule composition from the stability study of Example 4.

FIG. 13 shows the mean assay plot for the 5 mg capsule composition from the stability study of Example 4.

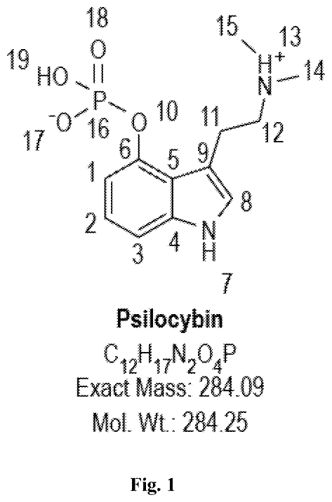

### DETAILED DESCRIPTION

### Definitions

Throughout this disclosure, various patents, patent applications and publications (including non-patent publications) are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “mass balance of psilocybin and related substances” as used herein means the sum of psilocybin and its related substances (i.e., psilocin and unidentified impurities defined by their relative retention times (RRT)) as determined by HPLC analysis. In some embodiments, the mass balance is determined on a weight % basis. In some embodiments, the unidentified impurities defined by their relative retention times are selected from the group consisting of about RRT 0.85, about RRT 1.25, about RRT 1.38, about RRT 1.44, and about RRT 1.51 as determined using the Related Substance HPLC Method. The following HPLC conditions (“Related Substance HPLC Method”) may be used to determine the mass balance of psilocybin and related substances of the composition of the present disclosure:

| Column | Waters Xbridge C18, | |
|---|---|---|
| | 3.5 | μm, |
| | 4.6 × 150 | mm |
| Column Temperature | 30° | C. |
| Flow Rate | 1.0 | mL/min |
| Wavelength (UV) | 267 | nm |
| Injection Volume | 20 | μL |
| Run Time | 27 | minutes |

| Mobile Phase A | Water:Methanol:TFA-95:5:0.1 | | |
|---|---|---|---|
| Mobile Phase B | Methanol:Water:TFA-95:5:0.1 | | |
| Preparation of diluent | Water:Methanol-95:5 | | |
| Gradient (% v/v) | Time (mins) | % A | % B |
| | 0 | 100 | 0 |
| | 2 | 100 | 0 |
| | 15 | 0 | 100 |
| | 20 | 0 | 100 |
| | 20.1 | 100 | 0 |
| | 27 | 100 | 0 |

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of psilocybin is that amount that is required to reduce at least one symptom of depression in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

As used herein, “substantially absent” with reference to XRPD diffractogram peak means the peak has a relative intensity compared to a reference peak present in the diffractogram of less than about 5%, less than about 4%, less than about 3%, less than about 2%, or less than about 1% of the intensity of the reference peak, or that the peak is not detectable.

As used herein the term “subject” and “patient” are used interchangeably.

The term “treating” as used herein with regard to a patient, refers to improving at least one symptom of the patient's disorder. Treating can be improving, or at least partially ameliorating a disorder.

The term “therapeutic effect” as used herein refers to a desired or beneficial effect provided by the method and/or the composition. For example, the method for treating depression provides a therapeutic effect when the method reduces at least one symptom of depression in a patient.

XRPD diffractograms and XRPD peak positions may be acquired using Cu Kα radiation.

DSC thermograms and TGA thermograms may be acquired using a heating rate of 20° C./min.

All disease and disorders listed herein are defined as described in the Diagnostic and Statistical Manual of Mental Disorders (DSM-5), published by the American Psychiatric Association, or in International Classification of Diseases (ICD), published by the World Health Organization.

As used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination.

The following abbreviations have been used herein:
DSC—Differential Scanning calorimetry
RT—room temperature
TBME—methyl tert-butyl ether
TGA—Thermogravimetric Analysis
THF—tetrahydrofuran
RRT—relative retention time
wrt—with respect to
XRPD—X-Ray Powder Diffraction

### Crystalline Forms of Psilocybin

The pharmaceutical compositions of the present disclosure can include one or more of the crystalline psilocybins disclosed in U.S. Pat. No. 10,519,175, which is herein incorporated by reference in its entirety for all purposes.

### Polymorph A

In some embodiments, the present disclosure provides pharmaceutical compositions wherein the psilocybin is crystalline Polymorph A (or “Polymorph A”).

According to the present disclosure, the psilocybin used is of a pharmaceutically acceptable chemical purity. In some embodiments, the psilocybin used is of a pharmaceutically acceptable chemical purity and is amorphous. In some embodiments, the psilocybin used is of a pharmaceutically acceptable chemical purity and is crystalline. In some embodiments, the psilocybin used is of a pharmaceutically acceptable chemical purity and is crystalline Polymorph A.

In some embodiments, the crystalline psilocybin is characterized by one or more of:
(a) peaks in an XRPD diffractogram at 11.5, 12.0 and 14.5° 2θ±0.1°2θ; and/or
(b) an endothermic event in a DSC thermogram having a first onset temperature of between 145° C. and 165° C. and a second onset temperature of between 205° C. and 220° C.

In some embodiments, the crystalline psilocybin is further characterized by at least one further peak at 19.7, 20.4, 22.2, 24.3 or 25.7° 2θ±0.1°20.

In some embodiments, the crystalline psilocybin is characterized by having either: (a) a water content of <0.5% w/w; or (b)<0.5% w/w loss in the TGA thermogram between 25° C. and 200° C.

In some embodiments, the psilocybin has a chemical purity of greater than 97% and no single impurity greater than 1% as determined by HPLC analysis. In some embodiments, the psilocybin has a chemical purity of greater than 97% by HPLC, and no single impurity of greater than 1% including phosphoric acid as measured by ³¹P NMR, and psilocin as determined by HPLC.

In some embodiments, the phosphoric acid impurity, and any other impurity that might be present are present in an amount of less than 0.5%, through less than 0.4%, 0.3%, to 0.2% or less.

In some embodiments, the psilocybin is Polymorph A and is characterized by at least one further peak in an XRPD diffractogram at 17.5, ° 2θ±0.1° 2θ which peak has a relative intensity compared to the peak at 14.5° 2θ±0.1° 2θ of at least 5%.

In some embodiments, the psilocybin is Polymorph A and is characterized by at least one further peak in an XRPD diffractogram at 17.5° 2θ±0.1°2θ which peak has a relative intensity compared to the peak at 14.5° 2θ±0.1° 2θ of at least 5%, at least 6%, at least 7%, through 8%, and 9% to at least 10%.

In some embodiments, the psilocybin is Polymorph A and is characterized by XRPD peaks at 11.5±0.1, 12.0±0.1, 14.5±0.1, 17.5±0.1, and 19.7±0.1° 2θ. In some embodiments, the psilocybin is Polymorph A and is characterized by XRPD peaks at 11.5±0.1, 12.0±0.1, 14.5±0.1, 17.5±0.1, and 19.7±0.1° 2θ and the psilocybin has a chemical purity of greater than 97% and no single impurity greater than 1% as determined by HPLC analysis.

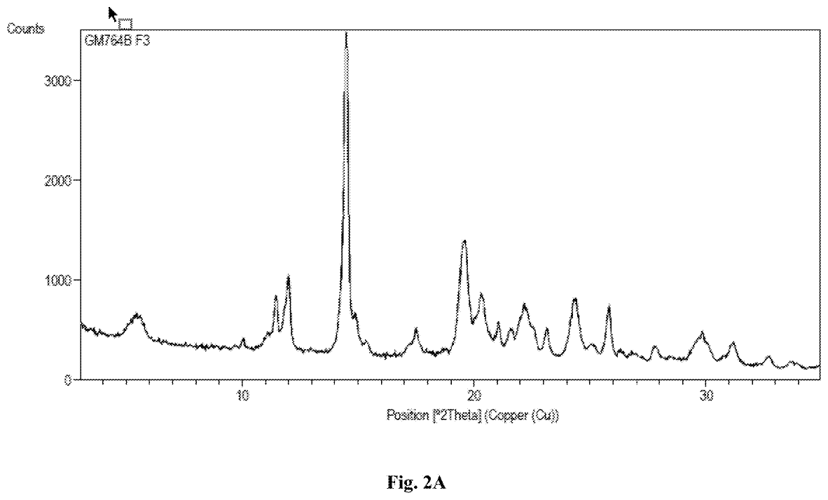

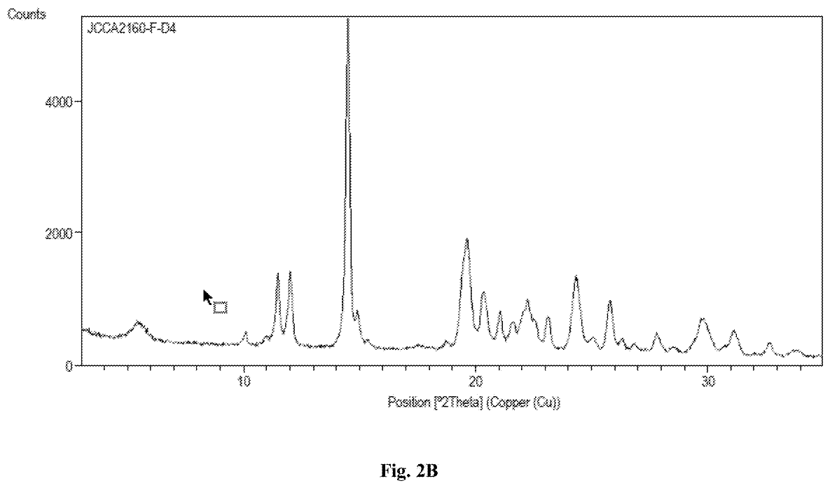

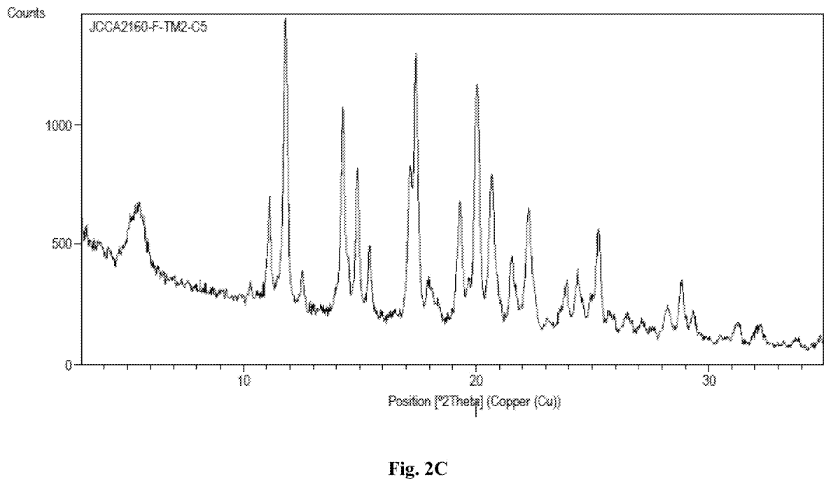

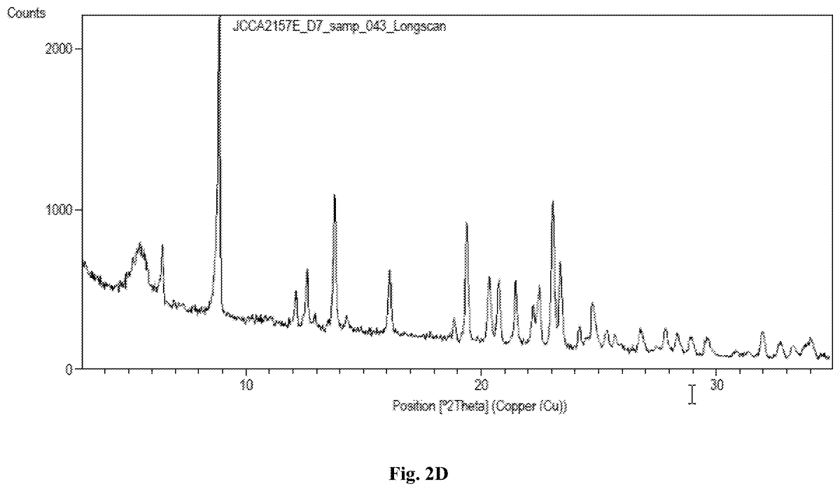

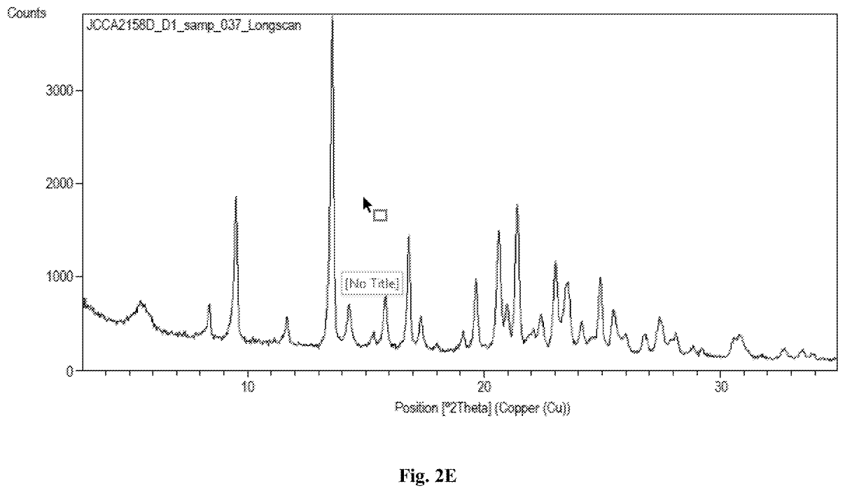

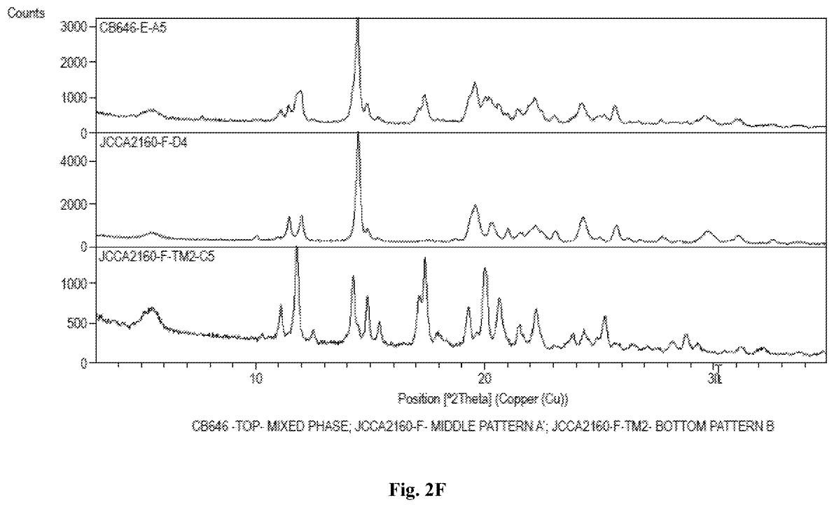

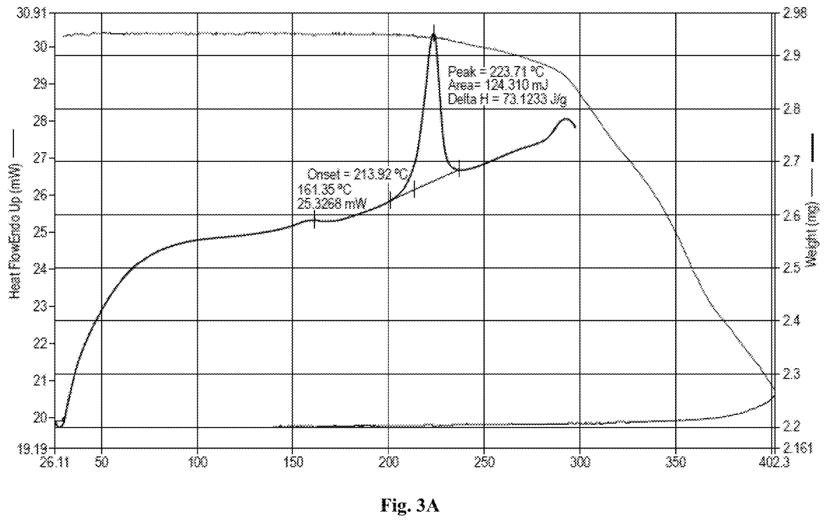

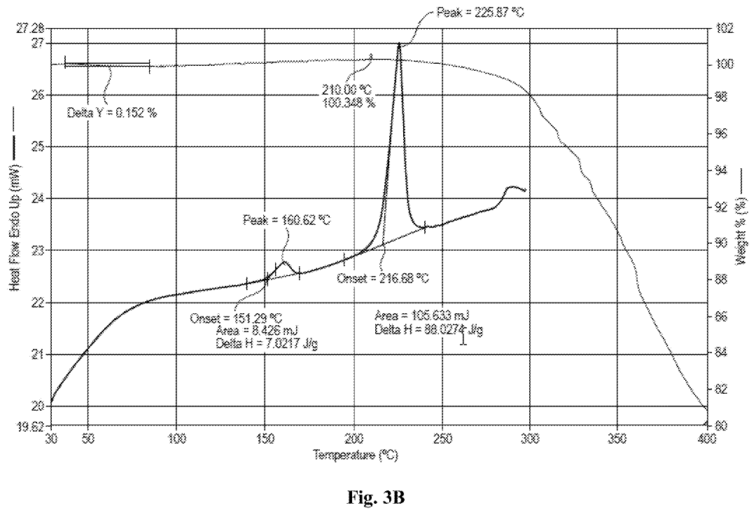

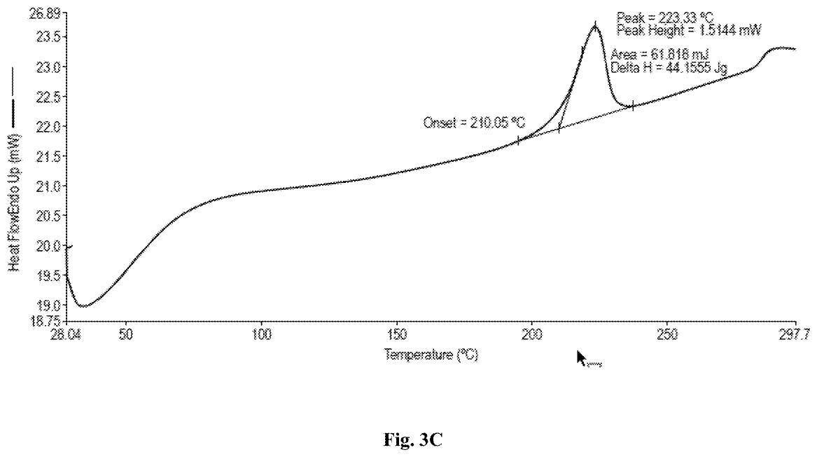

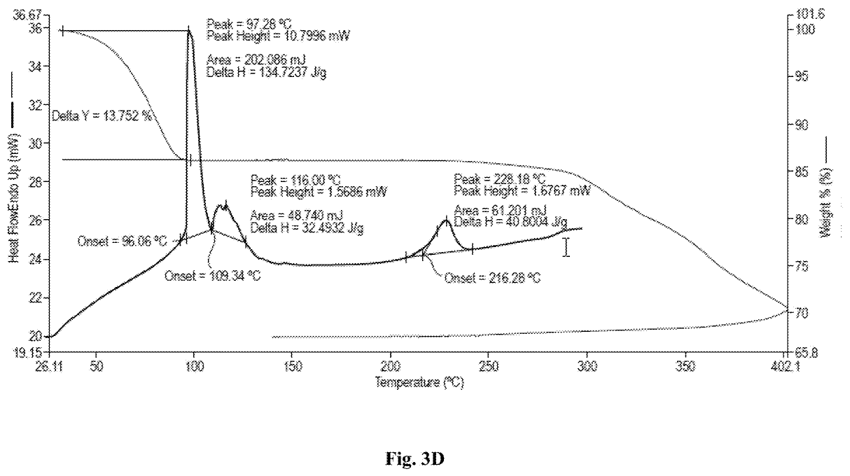

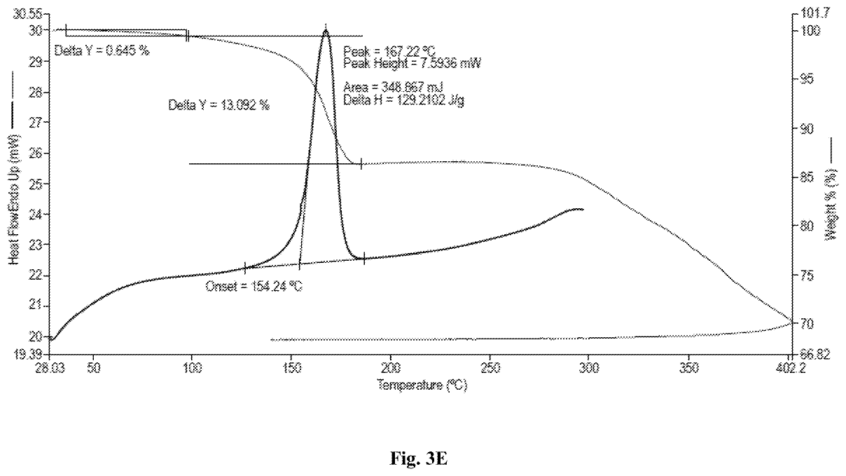

In some embodiments, the psilocybin is Polymorph A and has: (a) an XRPD diffractogram substantially as illustrated in FIG. 2A; and/or (b) a DSC thermogram substantially as illustrated in FIG. 3A.

In some embodiments, psilocybin Polymorph A has relatively large, uniform, rod shaped crystals with a size range of 50 to 200 microns.

In some embodiments disclosed herein, psilocybin Polymorph A exhibits an XRPD diffractogram characterized by the diffractogram summarized in Table 1. In some embodiments, described herein, the crystalline psilocybin Polymorph A comprises at least 3 peaks of (±0.1°2θ) of Table 1. In some embodiments, the crystalline psilocybin Polymorph A comprises at least 4 peaks of (±0.1°2θ) of Table 1. In some embodiments, the crystalline psilocybin Polymorph A comprises at least 5 peaks of (±0.1°2θ) of Table 1. In some embodiments, the crystalline psilocybin Polymorph A comprises at least 6 peaks of (±0.1°2θ) of Table 1. In some embodiments, the crystalline psilocybin Polymorph A comprises at least 8 peaks of (±0.1° 2θ) of Table 1. In some embodiments, the crystalline psilocybin Polymorph A comprises at least 10 peaks of (±0.1° 2θ) of Table 1. In some embodiments, the crystalline psilocybin Polymorph A comprising at least 15 peaks of (±0.1°2θ) of Table 1. A peak at about 17.5, ° 2θ±0.1° 2θ distinguishes psilocybin Polymorph A from Polymorph A′, in which the peak is absent or substantially absent (i.e. has a relative intensity compared to the peak at 14.5° 2θ±0.1° 2θ of less than 2%, or less than 1%).

TABLE 1
XRPD peak positions
for Polymorph A

| Position | Relative |
|---|---|
| [°2Θ] | Intensity [%] |
| 5.6 | 8.42 |
| 11.5 | 13.05 |
| 12.0 | 26.45 |
| 14.5 | 100 |
| 17.5 | 10.71 |
| 19.7 | 37.29 |
| 20.4 | 20.06 |
| 22.2 | 17.83 |
| 23.2 | 6.99 |
| 24.3 | 17.93 |
| 25.7 | 16.4 |
| 26.8 | 3.15 |
| 27.8 | 4.54 |
| 29.7 | 9.53 |
| 31.2 | 6.51 |
| 32.6 | 2.45 |
| 33.7 | 1.75 |

In some embodiments, crystalline psilocybin Polymorph A is characterized by XRPD diffractogram peaks at 11.5, 12.0, 14.5, and 17.5° 2θ±0.1°2θ. In some embodiments, crystalline psilocybin Polymorph A is further characterized by at least one additional peak appearing at 19.7, 20.4, 22.2, 24.3 or 25.7°2θ±0.1° 20. In some embodiments, crystalline psilocybin Polymorph A is further characterized by at least two additional peaks appearing at 19.7, 20.4, 22.2, 24.3 or 25.7° 2θ±0.1° 2θ. In some embodiments, crystalline psilocybin Polymorph A is further characterized by at least three additional peaks appearing at 19.7, 20.4, 22.2, 24.3 or 25.7° 2θ±0.1° 20. In yet a further embodiment, crystalline psilocybin Polymorph A exhibits an XRPD diffractogram substantially the same as the XRPD diffractogram shown in FIG. 2A.

In some embodiments, crystalline psilocybin Polymorph A is characterized by XRPD diffractogram peaks at 14.5 and 17.5° 2θ±0.1° 2θ with the peak at 17.5° 2θ having an intensity which is at least 5% of the intensity of the peak at 14.5° 20, or at least 6%, through at least 7%, at least 8%, at least 9%, to at least 10%.

In some embodiments, crystalline psilocybin Polymorph A is absent or substantially absent of an XRPD diffractogram peaks at 10.1. By “substantially absent” it is meant that any XRPD diffractogram peaks at 10.1 is less than 2% of the intensity of the peak at 14.5° 20, such as less than 1%, or is not detectable in the XRPD diffractogram,

In some embodiments, crystalline psilocybin Polymorph A is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C.

In some embodiments, crystalline psilocybin Polymorph A exhibits a DSC thermogram substantially the same as the DSC thermogram in FIG. 3A.

In some embodiments, crystalline psilocybin Polymorph A is characterized by having a water content of <0.5% w/w, such as <0.4% w/w, such as <0.3% w/w, such as <0.2% w/w, or such as <0.1% w/w. The skilled person would know of methods to determine the water content of a compound, for example Karl Fischer Titration. In some embodiments, crystalline psilocybin Polymorph A is characterized by having <0.5% w/w loss, such as <0.4% w/w, such as <0.3% w/w, such as <0.2% w/w, such as <0.1% w/w, in the TGA thermogram between ambient temperature, such as about 25° C., and 200° C. In some embodiments, crystalline psilocybin Polymorph A loses less than 2% by weight in a loss on drying test, such as less than 1% by weight, such as less than 0.5% by weight. The loss on drying test is performed at 70° C.

In some embodiments, crystalline psilocybin Polymorph A is a white to off white solid.

In some embodiments, crystalline psilocybin Polymorph A is of a pharmaceutically acceptable chemical purity, for example, the crystalline psilocybin Polymorph A has a chemical purity of greater than 97%, such as greater than 98%, or such as greater than 99% as determined by HPLC. In some embodiments, crystalline psilocybin Polymorph A has no single impurity of greater than 1%, greater than 0.5%, greater than 0.4%, though 0.3% to 0.2% or less including phosphoric acid as measured by ³¹P NMR, and psilocin as determined by HPLC. In some embodiments, crystalline psilocybin Polymorph A has a chemical purity of greater than 97 area %, greater than 98 area %, or greater than 99 area % as determined by HPLC. In some embodiments, crystalline psilocybin Polymorph A has no single impurity greater than 1 area %, greater than 0.5 area %, greater than 0.4 area %, though 0.3 area % to 0.2 area % or less, as determined by HPLC. In some embodiments, crystalline psilocybin Polymorph A has no single impurity greater than 2 area %, greater than 1.75 area %, greater than 1.5 area %, greater than 1.25 area %, greater than 1 area %, greater than 0.5 area %, greater than 0.4 area %, greater than 0.3 area %, greater than 0.2 area % or less, as determined by HPLC. In some embodiments, crystalline psilocybin Polymorph A does not contain psilocin at a level greater than 2 area %, greater than 1.75 area %, greater than 1.5 area %, greater than 1.25%, greater than 1 area %, greater than 0.5 area %, greater than 0.4 area %, greater than 0.3 area %, greater than 0.2 area % or less, as measured by HPLC. In some embodiments, crystalline psilocybin Polymorph A does not contain psilocin at a level greater than 1 area %, greater than 0.5 area %, greater than 0.4 area %, though 0.3 area % to 0.2 area % or less, as determined by HPLC. In some embodiments, crystalline psilocybin Polymorph A does not contain phosphoric acid at a level greater than 1 weight %, greater than 0.5 weight %, greater than 0.4 area %, though 0.3 area % to 0.2 area % or less, as determined by ³¹P NMR. In some embodiments, crystalline psilocybin Polymorph A has a chemical assay of at least 95 weight %, such as at least 96 weight %, or such as at least 98 weight %.

In some embodiments, crystalline psilocybin Polymorph A has no single impurity of greater than 2%, greater than 1.75%, greater than 1.5%, greater than 1.25%, greater than 1%, greater than 0.5%, greater than 0.4%, greater than 0.3%, or greater than 0.2% including phosphoric acid as determined by ³¹P NMR, and psilocin as determined by HPLC. In some embodiments, crystalline psilocybin Polymorph A does not contain psilocin at a level greater than 2%, greater than 1.75%, greater than 1.5%, greater than 1.25%, greater than 1%, greater than 0.5%, greater than 0.4%, greater than 0.3%, greater than 0.2% or less, as determined by HPLC. In some embodiments, crystalline psilocybin Polymorph A does not contain phosphoric acid at a level greater than 2 weight %, greater than 1.75 weight %, greater than 1.5 weight %, greater than 1.25 weight %, greater than 1 weight %, greater than 0.5 weight %, greater than 0.4 weight %, greater than 0.3 weight %, greater than 0.2 weight % or less, as determined by ³¹P NMR.

### Polymorph A′

Alternatively, the crystalline psilocybin may be in the form of Polymorph A′ characterized by one or more of:
(a) peaks in an XRPD diffractogram at 11.5, 12.0 and 14.5° 2θ±0.1° 20, but absent or substantially absent of a peak at 17.5° 2θ±0.1° 20;
(b) having a first onset temperature of between 145° C. and 165° C. and a second onset temperature of between 205° C. and 220° C.

By substantial absence of a peak at 17.5° 2θ±0.1° 2θ is meant that the peak at 17.5° 2θ±0.1°2θ (if present) has a relative intensity, compared to a peak at 14.5° 2θ±0.1°2θ, of less than 5%, in some embodiments, less than 4%, through less than 3%, to 2%, 1% or less.

In some embodiments, crystalline psilocybin Polymorph A′ exhibits an XRPD diffractogram characterized by the diffractogram summarized in Table 2. In some embodiments, described herein the crystalline psilocybin Polymorph A′ comprises at least 3 peaks of (±0.1° 2θ) of Table 2 but absent or substantially absent of a peak at 17.5° 2θ±0.1°2θ. In some embodiments, the crystalline psilocybin Polymorph A′ comprising at least 4 peaks of (±0.1°2θ) of Table 2 but absent or substantially absent of a peak at 17.5° 2θ±0.1° 20. In some embodiments, the crystalline psilocybin Polymorph A′ comprises at least 5 peaks of (±0.1°2θ) of Table 2 but absent or substantially absent of a peak at 17.5° 2θ±0.1°2θ. In some embodiments, the crystalline psilocybin Polymorph A′ comprises at least 6 peaks of (±0.1°2θ) of Table 2 but absent or substantially absent of a peak at 17.5° 2θ±0.1° 20. In some embodiments, the crystalline psilocybin Polymorph A′ comprises at least 8 peaks of (±0.1°2θ) of Table 2 but absent or substantially absent of a peak at 17.5° 2θ±0.1°2θ. In some embodiments, the crystalline psilocybin Polymorph A′ comprises at least 10 peaks of (±0.1°2θ) of Table 2 but absent or substantially absent of a peak at 17.5° 2θ±0.1°2θ. In some embodiments, the crystalline psilocybin Polymorph A′ comprises at least 15 peaks of (±0.1°2θ) of Table 2 but absent or substantially absent of a peak at 17.5° 2θ±0.1°2θ. In some embodiments, the crystalline psilocybin Polymorph A′ comprises at least 20 peaks of (±0.1°2θ) of Table 2 but the absence or substantial absence of a peak at 17.5° 2θ±0.1° 20. In some embodiments, the crystalline psilocybin Polymorph A′ comprises at least 25 peaks of (±0.1°2θ) of Table 2 but the peak at 17.5° 2θ±0.1° 2θ is absent or substantially absent.

TABLE 2
XRPD peak positions for Polymorph A′

| Position [°2Θ] | Relative Intensity [%] |
|---|---|
| 5.5 | 4.89 |
| 10.1 | 4.09 |
| 11.5 | 22.05 |
| 12.0 | 22.77 |
| 14.5 | 100 |
| 14.9 | 11.29 |
| 17.5 | 1.08 |
| 18.7 | 2.44 |
| 19.4 | 23.02 |
| 19.6 | 33.7 |
| 20.3 | 17.01 |
| 21.1 | 12.08 |
| 21.6 | 8.51 |
| 22.2 | 15.54 |
| 22.6 | 8.78 |
| 23.1 | 10.11 |
| 24.3 | 21.83 |
| 25.1 | 4.36 |
| 25.8 | 15.4 |
| 26.3 | 4.28 |
| 26.8 | 2.86 |
| 27.8 | 5.96 |
| 28.6 | 1.91 |
| 29.7 | 10.56 |
| 31.1 | 7.35 |
| 32.6 | 3.72 |
| 33.8 | 1.54 |

In some embodiments, crystalline psilocybin Polymorph A′ is characterized by XRPD diffractogram peaks at 11.5, 12.0, and 14.5° 2θ±0.1° 2θ but the substantial absence of a peak at 17.5° 2θ±0.1°2θ. In some embodiments, crystalline psilocybin Polymorph A′ is further characterized by at least one additional peak appearing at 19.7, 20.4, 22.2, 24.3, or 25.7° 2θ±0.1°2θ. In some embodiments, crystalline psilocybin Polymorph A′ is further characterized by at least two additional peaks appearing at 19.7, 20.4, 22.2, 24.3, or 25.7° 2θ±0.1°2θ. In some embodiments, crystalline psilocybin Polymorph A′ is further characterized, and distinguished from Polymorph A by the presence of a peak appearing at 10.1° 2θ±0.1°2θ. In yet a further embodiment, crystalline psilocybin Polymorph A′ exhibits an XRPD diffractogram substantially the same as the XRPD diffractogram shown in FIG. 2B.

In some embodiments, crystalline psilocybin Polymorph A′ is characterized by XRPD diffractogram peaks at 14.5 and 17.5° 2θ±0.1° 2θ wherein the intensity of the peak at 17.5° 2θ is less than 5% of the intensity of the peak at 14.5° 20, such as less than 4%, such as less than 3%, such as at less than 2%, such as less than 1%, or such as about 1%.

In some embodiments, crystalline psilocybin Polymorph A′ is characterized by XRPD diffractogram peaks at 10.1 and 14.5° 2θ±0.1° 2θ wherein the intensity of the peak at 10.1°2θ is at least 1% of the intensity of the peak at 14.5° 20, such as at least than 2%, such as at least than 3%, or such as about 4%.

In some embodiments, crystalline psilocybin Polymorph A′ is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A′ exhibits a DSC thermogram substantially the same as the DSC thermogram in FIG. 3B.

In some embodiments, crystalline psilocybin Polymorph A′ is characterized by having a water content of <0.5% w/w, such as <0.4% w/w, such as <0.3% w/w, such as <0.2% w/w, or such as <0.1% w/w. The skilled person would know of methods to determine the water content of a compound, for example Karl Fischer Titration. In some embodiments, crystalline psilocybin Polymorph A′ is characterized by having <0.5% w/w loss, such as <0.4% w/w, such as <0.3% w/w, such as <0.2% w/w, such as <0.1% w/w, in the TGA thermogram between ambient temperature, such as 25° C., and 200° C. In some embodiments, crystalline psilocybin Polymorph A′ loses less than 2% by weight in a loss on drying test, such as less than 1% by weight, such as less than 0.5% by weight. The loss on drying test is performed at 70° C.

In some embodiments, crystalline psilocybin Polymorph A′ is a white to off white solid.

XRPD diffractograms and XRPD peak positions are acquired using Cu Kα radiation.

DSC and TGA thermograms are acquired using a heating rate of 20° C./min.

In some embodiments, there is provided high purity crystalline psilocybin, Polymorph A or Polymorph A′, exhibiting an XRPD diffractogram as substantially illustrated in FIG. 2A or 2B and a DSC thermograph as substantially illustrated in FIG. 3A or 3B or a mixture thereof.

In some embodiments, the crystalline psilocybin Polymorph A exhibits an XRPD diffractogram as illustrated in FIG. 2A and a DSC thermograph as illustrated in FIG. 3A.

In some embodiments, the crystalline psilocybin Polymorph A′ exhibits an XRPD diffractogram as substantially illustrated in FIG. 2B and a DSC thermograph as substantially illustrated in FIG. 3B.

In some embodiments, the high purity crystalline psilocybin Polymorph A is characterized by a XRPD diffractogram as substantially illustrated in FIG. 2A and a DSC thermograph as substantially illustrated in FIG. 3A.

In some embodiments, the high purity crystalline psilocybin Polymorph A′ is characterized by a XRPD diffractogram as illustrated in FIG. 2B and a DSC thermograph as illustrated in FIG. 3B.

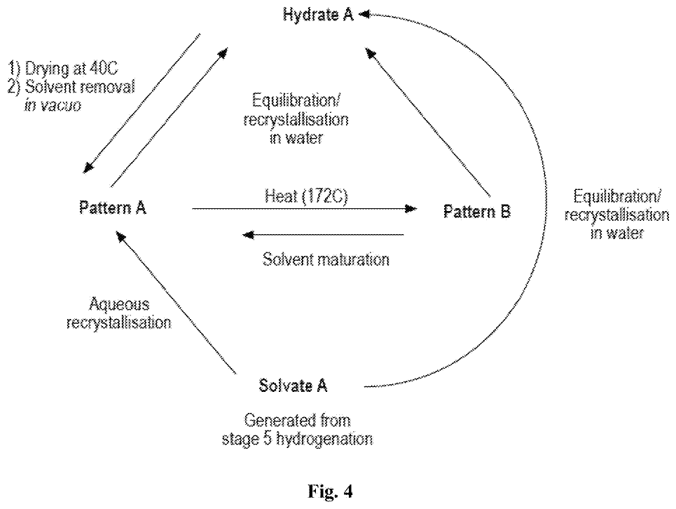

Polymorph A (including its isostructural variant Polymorph A′) (FIGS. 2A and 2B) differs from Polymorph B (FIG. 2C), the Hydrate A (FIG. 2D) and the ethanol solvate (FIG. 2E: Solvate A), and the relationship between some of the different forms is illustrated in FIG. 4.

The crystalline psilocybin Polymorph A′ is a white to off white solid, and/or has a chemical purity of greater than 97%, in some embodiments greater than 98%, and in some embodiments greater than 99% by HPLC, and has no single impurity of greater than 2%, greater than 1.75%, greater than 1.5%, greater than 1.25%, greater than 1%, greater than 0.75%, greater than 0.5%, greater than 0.4%, through 0.3% to 0.2% or less including phosphoric acid as determined by ³¹P NMR, and psilocin as determined by HPLC. In some embodiments, crystalline psilocybin Polymorph A′, has a chemical assay of at least 95 weight %, such as at least 96 weight %, or such as at least 98 weight %.

In some embodiments, crystalline psilocybin Polymorph A′ is of a pharmaceutically acceptable chemical purity, for example, the crystalline psilocybin Polymorph A′ has a chemical purity of greater than 97%, such as greater than 98%, or such as greater than 99%, as measured by HPLC. In some embodiments, crystalline psilocybin Polymorph A′ has no single impurity of greater than 2%, greater than 1.75%, greater than 1.5%, greater than 1.25%, greater than 1%, greater than 0.5%, greater than 0.4%, greater than 0.3%, greater than 0.2% or less including phosphoric acid as determined by ³¹P NMR, and psilocin as determined by HPLC. In some embodiments, crystalline psilocybin Polymorph A′ has no single impurity greater than 2 area %, greater than 1.75 area %, greater than 1.5 area %, greater than 1.25 area %, greater than 1 area %, greater than 0.5 area %, greater than 0.4 area %, greater than 0.3 area %, greater than 0.2 area % or less, as determined by HPLC. In some embodiments, crystalline psilocybin Polymorph A′ does not contain psilocin at a level greater than 2%, greater than 1.75%, greater than 1.5%, greater than 1.25%, greater than 1%, greater than 0.5%, greater than 0.4%, greater than 0.3%, greater than 0.2% or less, as determined by HPLC. In some embodiments, crystalline psilocybin Polymorph A′ does not contain psilocin at a level greater than 2 area %, greater than 1.75 area %, greater than 1.5 area %, greater than 1.25%, greater than 1 area %, greater than 0.5 area %, greater than 0.4 area %, greater than 0.3 area %, greater than 0.2 area % or less, as determined by HPLC. In some embodiments, crystalline psilocybin Polymorph A′ does not contain phosphoric acid at a level greater than 2 weight %, greater than 1.75 weight %, greater than 1.5 weight %, greater than 1.25 weight %, greater than 1 weight %, greater than 0.5 weight %, greater than 0.4 weight %, greater than 0.3 weight %, greater than 0.2 weight % or less, as measured by ³¹P NMR.

The heating of Polymorph A or A′ results in an endothermic event having an onset temperature of circa 150° C. corresponding to solid-solid transition of Polymorph A or Polymorph A′ to Polymorph B. Continued heating of the resulting solid, i.e., Polymorph B, results in a second endothermic event corresponding to a melting point having an onset temperature of between 205 and 220° C. (see FIGS. 3A and 3B).

### Hydrate A

In some embodiments there is provided a crystalline form of psilocybin, Hydrate A, characterized by one or more of:
(a) peaks in an XRPD diffractogram at 8.9, 12.6 and 13.8° 2θ±0.1°2θ; and/or
(b) an endothermic event in a DSC thermogram having a first onset temperature of between 90° C. and 100° C., a second onset temperature of between 100° C. and 120° C. and a third onset temperature of between 210° C. and 220° C.

The XPRD diffractogram may exhibit at least one further peak at 6.5, 12.2, 19.4, 20.4 or 20.8° 2θ±0.1° 2θ.

In some embodiments, psilocybin Hydrate A exhibits an XRPD diffractogram characterized by the diffractogram summarized in Table 3. In some embodiments, described herein the crystalline psilocybin Hydrate A comprises at least 3 peaks of (±0.1°2θ) of Table 3. In some embodiments, the crystalline psilocybin Hydrate A comprises at least 4 peaks of (±0.1°2θ) of Table 3. In some embodiments, the crystalline psilocybin Hydrate A comprises at least 5 peaks of (±0.1°2θ) of Table 3. In some embodiments, the crystalline psilocybin Hydrate A comprises at least 8 peaks of (±0.1°2θ) of Table 3. In some embodiments, the crystalline psilocybin Hydrate A comprises at least 10 peaks of (±0.1°2θ) of Table 3.

TABLE 3
XRPD peak positions for Hydrate A

| Position [°2Θ] | Relative Intensity [%] |
|---|---|
| 5.6 | 14.4 |
| 6.5 | 18.84 |
| 8.9 | 100 |
| 12.2 | 11.51 |
| 12.6 | 18.65 |
| 13.8 | 44.22 |
| 16.2 | 21.22 |
| 18.9 | 6.62 |
| 19.4 | 38.68 |
| 20.4 | 21.32 |
| 20.8 | 19.73 |
| 21.5 | 20.75 |
| 22.3 | 12.8 |
| 22.5 | 19.38 |
| 23.1 | 47.53 |
| 23.5 | 25.79 |
| 24.3 | 5.62 |
| 24.8 | 14.62 |
| 25.4 | 5.27 |
| 26.9 | 6.53 |
| 27.9 | 7.82 |
| 28.4 | 5.78 |
| 29.0 | 5.09 |
| 29.7 | 4.83 |
| 32.1 | 8.27 |
| 32.8 | 4.81 |
| 33.4 | 3.74 |
| 34.2 | 5.96 |

In some embodiments, crystalline psilocybin Hydrate A is characterized by XRPD diffractogram peaks at 8.9, 12.6 and 13.8° 2θ±0.1°2θ. In some embodiments, crystalline psilocybin Hydrate A is further characterized by at least one peak appearing at 6.5, 12.2, 19.4, 20.4 or 20.8° 2θ±0.1°2θ. In some embodiments, crystalline psilocybin Hydrate A is further characterized by at least two peaks appearing at 6.5, 12.2, 19.4, 20.4 or 20.8° 2θ±0.1° 20. In yet a further embodiment, crystalline psilocybin Hydrate A exhibits an XRPD diffractogram substantially the same as the XRPD diffractogram shown in FIG. 2D.

In some embodiments, crystalline psilocybin Hydrate A is characterized by an endothermic event in a DSC thermogram having a first onset temperature of between 85° C. and 105° C., such as between 90° C. and 100° C., such as at about 96° C.; a second onset temperature of between 100° C. and 120° C. such as between 105° C. and 115° C., such as at about 109° C.; and a third onset temperature of between 205 and 220° C., such as between 21° and 220° C., such as between 21° and 218° C., or such as between 21° and 216° C., such as about 216° C. In some embodiments, crystalline psilocybin Hydrate A exhibits a DSC thermogram substantially the same as the DSC thermogram in FIG. 3D.

In some embodiments, crystalline psilocybin Hydrate A is characterized by having a water content of between 10% and 18%, such as between 12% and 16%, or such as about 13%. The skilled person would know of methods to determine the water content of a compound, for example Karl Fischer Titration. In some embodiments, crystalline psilocybin Hydrate A is characterized by having a weight loss in the TGA thermogram of between 10% and 18%, such as between 12% and 16%, or such as about 13%, between ambient temperature, such as about 25° C., to 120° C.

In some embodiments, crystalline psilocybin Hydrate A is of a pharmaceutically acceptable chemical purity, for example, the crystalline psilocybin Hydrate A has a chemical purity of greater than 97%, such as greater than 98%, or such as greater than 99% as determined by HPLC. In some embodiments, crystalline psilocybin Hydrate A has no single impurity of greater than 1%, greater than 0.5%, greater than 0.4%, though 0.3% to 0.2% or less including phosphoric acid as measured by ³¹P NMR, and psilocin as determined by HPLC. In some embodiments, crystalline psilocybin Hydrate A has a chemical purity of greater than 97 area %, greater than 98 area %, or greater than 99 area % as determined by HPLC. In some embodiments, crystalline psilocybin Hydrate A has no single impurity greater than 1 area %, greater than 0.5 area %, greater than 0.4 area %, though 0.3 area % to 0.2 area % or less, as determined by HPLC. In some embodiments, crystalline psilocybin Hydrate A has no single impurity greater than 2 area %, greater than 1.75 area %, greater than 1.5 area %, greater than 1.25 area %, greater than 1 area %, greater than 0.5 area %, greater than 0.4 area %, greater than 0.3 area %, greater than 0.2 area % or less, as determined by HPLC. In some embodiments, crystalline psilocybin Hydrate A does not contain psilocin at a level greater than 2 area %, greater than 1.75 area %, greater than 1.5 area %, greater than 1.25%, greater than 1 area %, greater than 0.5 area %, greater than 0.4 area %, greater than 0.3 area %, greater than 0.2 area % or less, as measured by HPLC. In some embodiments, crystalline psilocybin Hydrate A does not contain psilocin at a level greater than 1 area %, greater than 0.5 area %, greater than 0.4 area %, though 0.3 area % to 0.2 area % or less, as determined by HPLC. In some embodiments, crystalline psilocybin Hydrate A does not contain phosphoric acid at a level greater than 1 weight %, greater than 0.5 weight %, greater than 0.4 area %, though 0.3 area % to 0.2 area % or less, as determined by ³¹P NMR. In some embodiments, crystalline psilocybin Hydrate A has a chemical assay of at least 95 weight %, such as at least 96 weight %, or such as at least 98 weight %.

In some embodiments, crystalline psilocybin Hydrate A has no single impurity of greater than 2%, greater than 1.75%, greater than 1.5%, greater than 1.25%, greater than 1%, greater than 0.5%, greater than 0.4%, greater than 0.3%, or greater than 0.2% including phosphoric acid as determined by ³¹P NMR, and psilocin as determined by HPLC. In some embodiments, crystalline psilocybin Hydrate A does not contain psilocin at a level greater than 2%, greater than 1.75%, greater than 1.5%, greater than 1.25%, greater than 1%, greater than 0.5%, greater than 0.4%, greater than 0.3%, greater than 0.2% or less, as determined by HPLC. In some embodiments, crystalline psilocybin Hydrate A does not contain phosphoric acid at a level greater than 2 weight %, greater than 1.75 weight %, greater than 1.5 weight %, greater than 1.25 weight %, greater than 1 weight %, greater than 0.5 weight %, greater than 0.4 weight %, greater than 0.3 weight %, greater than 0.2 weight % or less, as determined by ³¹P NMR.

### Polymorph B

In some embodiments, there is provided a crystalline form of psilocybin, Polymorph B, characterized by one or more of:
(a) peaks in an XRPD diffractogram at 11.1, 11.8 and 14.3°2θ±0.1° 20; and/or
(b) a single endothermic event in a DSC thermogram having an onset temperature of between 205 and 220° C.

The XPRD diffractogram may exhibit at least one further peak at 14.9, 15.4, 19.3, 20.0 or 20.6° 2θ±0.1° 2θ.

In some embodiments, the XRPD diffractogram is as substantially illustrated in FIG. 2C.

In some embodiments, the DSC thermogram is substantially as illustrated in FIG. 3C.

In some embodiments, psilocybin Polymorph B exhibits an XRPD diffractogram characterized by the diffractogram summarized in Table 4. In some embodiments, described herein the crystalline psilocybin Polymorph B comprises at least 3 peaks of (±0.1°2θ) of Table 4. In some embodiments, the crystalline psilocybin Polymorph B comprises at least 4 peaks of (±0.1°2θ) of Table 4. In some embodiments, the crystalline psilocybin Polymorph B comprises at least 5 peaks of (±0.1°2θ) of Table 4. In some embodiments, the crystalline psilocybin Polymorph B comprising at least 8 peaks of (±0.1° 2θ) of Table 4. In some embodiments, the crystalline psilocybin Polymorph B comprises at least 10 peaks of (±0.1° 2θ) of Table 4.

TABLE 4
XRPD peak positions for Polymorph B

| Position [°2Θ] | Relative Intensity [%] |
|---|---|
| 5.5 | 21.33 |
| 11.1 | 36.91 |
| 11.8 | 100.00 |
| 12.5 | 12.73 |
| 14.3 | 70.23 |
| 14.9 | 50.01 |
| 15.4 | 23.67 |
| 17.1 | 51.58 |
| 17.4 | 91.25 |
| 18.0 | 12.61 |
| 19.3 | 39.33 |
| 20.0 | 76.61 |
| 20.6 | 50.26 |
| 21.5 | 20.77 |
| 22.3 | 40.19 |
| 23.9 | 13.32 |
| 24.3 | 16.03 |
| 25.3 | 32.94 |
| 28.3 | 7.60 |
| 28.9 | 17.89 |
| 29.3 | 8.96 |
| 31.3 | 6.57 |
| 32.2 | 6.90 |
| 33.8 | 2.37 |

In some embodiments, crystalline psilocybin Polymorph B is characterized by XRPD diffractogram peaks at 11.1, 11.8 and 14.3°2θ±0.1°2θ. In some embodiments, crystalline psilocybin Polymorph B is further characterized by at least one peak appearing at 14.9, 15.4, 19.3, 20.0 or 20.6° 2θ±0.1°2θ. In some embodiments, crystalline psilocybin Polymorph B is further characterized by at least two peaks appearing at 14.9, 15.4, 19.3, 20.0 or 20.6° 2θ±0.1° 20. In yet a further embodiment, crystalline psilocybin Polymorph B exhibits an XRPD diffractogram substantially the same as the XRPD diffractogram shown in FIG. 2C.

In some embodiments, crystalline psilocybin Polymorph B is characterized by a single endothermic event in a DSC thermogram having an onset temperature of between 205 and 220° C., such as between 21° and 220° C., such as between 21° and 218° C., or such as between 21° and 216° C. In some embodiments, crystalline psilocybin Polymorph B exhibits a DSC thermogram substantially the same as the DSC thermogram in FIG. 3C.

In some embodiments, crystalline psilocybin Polymorph B is characterized by having a water content of <0.5% w/w, such as <0.4% w/w, such as <0.3% w/w, such as <0.2% w/w, or such as <0.1% w/w. The skilled person would know of methods to determine the water content of a compound, for example Karl Fischer Titration. In some embodiments, crystalline psilocybin Polymorph B is characterized by having <0.5% w/w loss, such as <0.4% w/w, such as <0.3% w/w, such as <0.2% w/w, such as <0.1% w/w, in the TGA thermogram between ambient temperature, such as about 25° C., and 200° C. In some embodiments, crystalline psilocybin Polymorph B loses less than 2% by weight in a loss on drying test, such as less than 1% by weight, such as less than 0.5% by weight. The loss on drying test is performed at 70° C.

In some embodiments, crystalline psilocybin Polymorph B is of a pharmaceutically acceptable chemical purity, for example, the crystalline psilocybin Polymorph B has a chemical purity of greater than 97%, such as greater than 98%, or such as greater than 99% as determined by HPLC. In some embodiments, crystalline psilocybin Polymorph B has no single impurity of greater than 1%, greater than 0.5%, greater than 0.4%, though 0.3% to 0.2% or less including phosphoric acid as measured by ³¹P NMR, and psilocin as determined by HPLC. In some embodiments, crystalline psilocybin Polymorph B has a chemical purity of greater than 97 area %, greater than 98 area %, or greater than 99 area % as determined by HPLC. In some embodiments, crystalline psilocybin Polymorph B has no single impurity greater than 1 area %, greater than 0.5 area %, greater than 0.4 area %, though 0.3 area % to 0.2 area % or less, as determined by HPLC. In some embodiments, crystalline psilocybin Polymorph B has no single impurity greater than 2 area %, greater than 1.75 area %, greater than 1.5 area %, greater than 1.25 area %, greater than 1 area %, greater than 0.5 area %, greater than 0.4 area %, greater than 0.3 area %, greater than 0.2 area % or less, as determined by HPLC. In some embodiments, crystalline psilocybin Polymorph B does not contain psilocin at a level greater than 2 area %, greater than 1.75 area %, greater than 1.5 area %, greater than 1.25%, greater than 1 area %, greater than 0.5 area %, greater than 0.4 area %, greater than 0.3 area %, greater than 0.2 area % or less, as measured by HPLC. In some embodiments, crystalline psilocybin Polymorph B does not contain psilocin at a level greater than 1 area %, greater than 0.5 area %, greater than 0.4 area %, though 0.3 area % to 0.2 area % or less, as determined by HPLC. In some embodiments, crystalline psilocybin Polymorph B does not contain phosphoric acid at a level greater than 1 weight %, greater than 0.5 weight %, greater than 0.4 area %, though 0.3 area % to 0.2 area % or less, as determined by ³¹P NMR. In some embodiments, crystalline psilocybin Polymorph B has a chemical assay of at least 95 weight %, such as at least 96 weight %, or such as at least 98 weight %.

In some embodiments, crystalline psilocybin Polymorph B has no single impurity of greater than 2%, greater than 1.75%, greater than 1.5%, greater than 1.25%, greater than 1%, greater than 0.5%, greater than 0.4%, greater than 0.3%, or greater than 0.2% including phosphoric acid as determined by ³¹P NMR, and psilocin as determined by HPLC. In some embodiments, crystalline psilocybin Polymorph B does not contain psilocin at a level greater than 2%, greater than 1.75%, greater than 1.5%, greater than 1.25%, greater than 1%, greater than 0.5%, greater than 0.4%, greater than 0.3%, greater than 0.2% or less, as determined by HPLC. In some embodiments, crystalline psilocybin Polymorph B does not contain phosphoric acid at a level greater than 2 weight %, greater than 1.75 weight %, greater than 1.5 weight %, greater than 1.25 weight %, greater than 1 weight %, greater than 0.5 weight %, greater than 0.4 weight %, greater than 0.3 weight %, greater than 0.2 weight % or less, as determined by ³¹P NMR.

The psilocybin in the form Polymorph A or A′ has the general properties illustrated in Table 5 below:

TABLE 5

| | |
|---|---|
| Appearance: | White to off white solid |
| Major endothermic event in | 210-215° C. |
| DSC (onset temperature) | |
| (corresponding to a melt): | |
| Hygroscopicity: | Psilocybin forms Hydrate A at high humidity |
| | and when added to water but the water of |
| | hydration is lost rapidly on drying. The |
| | anhydrous form is therefore being developed. |
| Crystalline form: | Anhydrous Polymorph A and/ or A′ |
| pKa (calculated): | 1.74, 6.71, 9.75 |
| Solubility | approx. 15 mg/ml in Water |

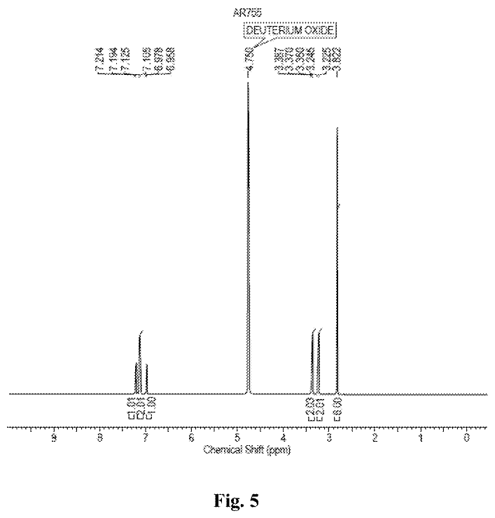

The psilocybin conforms to the spectra as set out in Table 6 below and illustrated in the spectra of FIGS. 5-8.

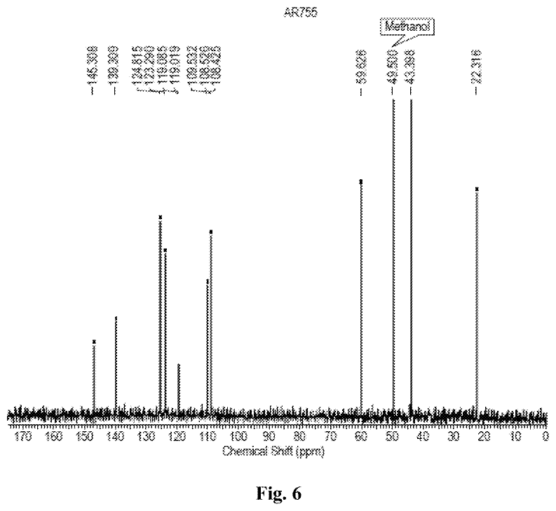

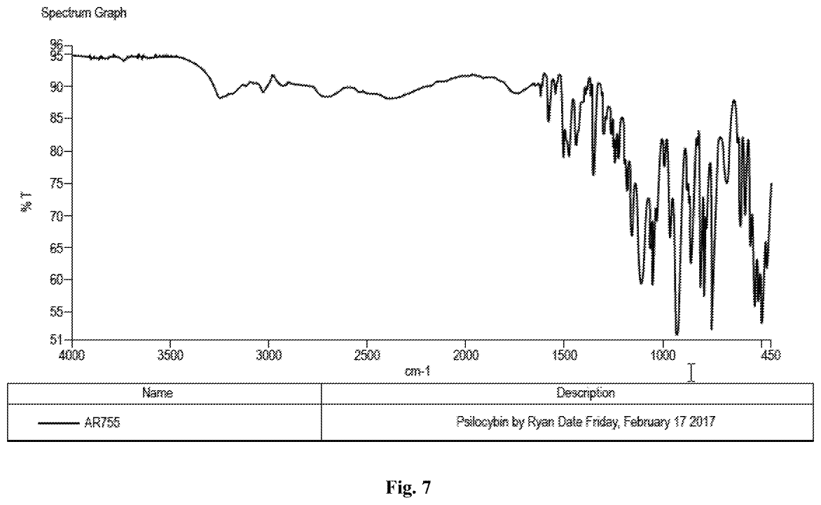

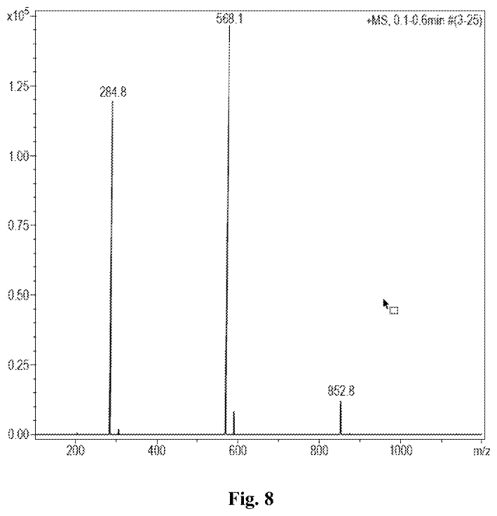

TABLE 6

| Technique | Conclusions |
|---|---|
| Proton (¹H) and Carbon | Assignment of the proton (FIG. 5) and |
| (¹³C) NMR | carbon spectra (FIG. 6) are concordant with |
| | Psilocybin. |
| FT-Infrared | Assignment of the FT-IR spectrum (FIG. 7) |
| Spectroscopy (FT-IR) | is concordant with Psilocybin. |
| Mass Spectroscopy (MS) | Assignment of the mass spectrum (FIG. 8) is |
| | concordant with Psilocybin. |

The high purity is attained by careful control of reaction conditions to ensure that potential organic impurities are significantly reduced.

Known and potential impurities in Psilocybin are shown in Table 7 below:

TABLE 7

| | Relative | | |
|---|---|---|---|
| | Retention | | |
| | Time | | |
| Impurity | (RRT) | Structure | Origin |
| Psilocin | 1.65 | 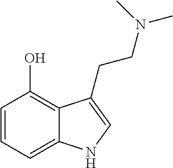 | Starting material (stage 3). Also generated by hydrolysis of Psilocybin. Only significant impurity observed in psilocybin batches. |
| Stage 4A | | 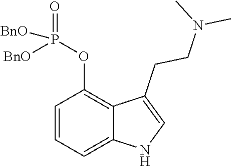 [SMILES: CN(C)CCc1c[nH]c2cccc(OP(=O)(OCc3ccccc3)OCc3ccccc3)c12] | Initial product formed in the stage 4 reaction. Converts to stage 4 on stirring in THF. Converts to Psilocybin in stage 5. |
| Stage 4 | 2.74 | 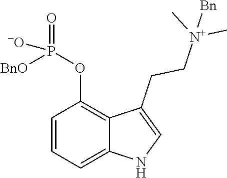 | Intermediate |
| N-Benzylated stage 4 | | 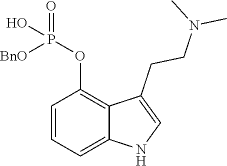 [SMILES: CN(C)CCc1c[nH]c2cccc(OP(=O)(O)OCc3ccccc3)c12] | Identified by MS in Stage 4. Converts to Psilocybin in stage 5. |
| Stage 4 Anhydride Impurity | | 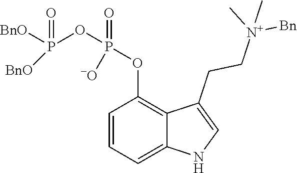 | Identified by MS in Stage 4. Converts to Stage 5 Pyrophosphoric acid impurity in stage 5. |
| Stage 5 Pyrophosphoric acid impurity | | 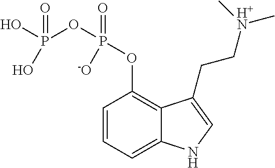 | Identified by MS Formed from the stage 4 anhydride. Removed in the stage 6 re-crystallisation by a combination of hydrolysis to Psilocybin and increased solubility due to the extra phosphate group. |
| Stage 5 (intermediates) | 1.89 and 2.45 | 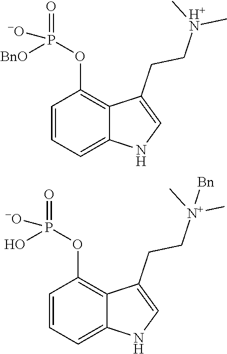 | 2 Intermediates are formed during the hydrogenation. These subsequently convert to product (structures based on chemistry). Monitored and controlled in stage 5 reaction. |

### Pharmaceutical Compositions of the Present Disclosure

The present disclosure provides pharmaceutical compositions containing psilocybin that are stable when stored for prolonged periods of time.

In some embodiments, the present disclosure provides a pharmaceutical composition comprising: a therapeutically effective amount of psilocybin; and one or more pharmaceutically acceptable excipients, wherein potency of the psilocybin in the composition and the mass balance of psilocybin and related substances in the composition remains at industry and pharmaceutically acceptable levels (e.g., European Pharmacopeia, USP) under industry and pharmaceutically acceptable storage conditions (e.g., European Pharmacopeia, USP) and the psilocybin is provided in pharmaceutically acceptable content uniformity (as measured by European Pharmacopeia 2.96. or USP <905>).

In some embodiments, the present disclosure provides a pharmaceutical composition comprising: a therapeutically effective amount of psilocybin; and one or more pharmaceutically acceptable excipients, wherein, after storage of the composition at 40° C. and 75% relative humidity for one month, the potency of the psilocybin in the composition decreases by less than 5% and the mass balance of psilocybin and related substances is greater than 97%.

In some embodiments, after storage of the composition at 40° C. and 75% relative humidity for two months, three months, four months, five months, or six months the potency of the psilocybin in the composition decreases by less than 5% and the mass balance of psilocybin and related substances is greater than 97%. In some embodiments, after storage of the composition at 40° C. and 75% relative humidity for two months, the potency of the psilocybin in the composition decreases by less than 5% and the mass balance of psilocybin and related substances is greater than 97%. In some embodiments, after storage of the composition at 40° C. and 75% relative humidity for the disclosed period of time, the potency of the psilocybin in the composition decreases by less than 5%, less than 4%, less than 2%, or less than 1% and the mass balance of the psilocybin and related substances in the composition is greater than 97%. In some embodiments, after storage of the composition at 40° C. and 75% relative humidity for the disclosed period of time, the potency of the psilocybin in the composition decreases by less than 5%, less than 4%, less than 2%, or less than 1% and the mass balance of the psilocybin and related substances in the composition is greater than 98% or 99%.

In some embodiments, the composition comprises about 1% to 10% by weight of psilocybin, e.g., about 1%, about 2%, about 3%, about 4%, about 5%, about 6%, about 7%, about 8%, about 9%, or about 10%, including all ranges and values therebetween. In some embodiments, the composition comprises about 1% to 15% by weight of psilocybin, e.g., about 1%, about 2%, about 3%, about 4%, about 5%, about 6%, about 7%, about 8%, about 9%, about 10%, about 11%, about 12%, about 13%, about 14% or about 15%, including all ranges and values therebetween. In some embodiments, the composition comprises about 1 mg to 40 mg of psilocybin, e.g., 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg, including all ranges and values therebetween. In some embodiments, the composition comprises about 1 mg to 25 mg of psilocybin, e.g., 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 22 mg, 24 mg, or 25 mg, including all ranges and values therebetween. In some embodiments, the composition comprises about 1 mg to 10 mg of psilocybin, e.g., 1 mg, 2 mg, 3 mg, 4 mg, 5 mg, 6 mg, 7 mg, 8 mg, 9 mg, or 10 mg, including all ranges and values therebetween. In some embodiments, the composition comprises about 1 mg to 5 mg of psilocybin, e.g., 1 mg, 2 mg, 3 mg, 4 mg, or 5 mg, including all ranges and values therebetween. In some embodiments, the composition comprises about 1 mg of psilocybin. In some embodiments, the composition comprises about 5 mg of psilocybin. In some embodiments, the composition comprises 10 mg of psilocybin. In some embodiments, the composition comprises 15 mg of psilocybin. In some embodiments, the composition comprises about 20 mg of psilocybin. In some embodiments, the composition comprises about 25 mg of psilocybin.

In some embodiments, the chemical purity of the psilocybin in the composition is not less than 97%, not less than 98%, or not less than 99%, as determined by HPLC analysis. In some embodiments, the chemical purity of the psilocybin in the composition is not less than 97%, as determined by HPLC analysis.

In some embodiments, the chemical purity of the psilocybin in the composition is greater than 97%, greater than 98%, or greater than 99%, as determined by HPLC analysis. In some embodiments, the chemical purity of the psilocybin in the composition is greater than 97%, as determined by HPLC analysis. In some embodiments, the chemical purity of the psilocybin in the composition is greater than 97% and no single impurity of greater than 2% as determined by HPLC analysis. In some embodiments, the chemical purity of the psilocybin in the composition is greater than 97% and no single impurity of greater than 1% as determined by HPLC analysis.

In some embodiments, the psilocybin comprises a crystalline psilocybin. In some embodiments, the crystalline psilocybin is a crystalline psilocybin disclosed in U.S. Pat. No. 10,519,175, which is herein incorporated by reference in its entirety for all purposes. In some embodiments, the crystalline psilocybin is crystalline psilocybin Polymorph A. In some embodiments, the crystalline psilocybin is crystalline psilocybin Hydrate A. In some embodiments, the crystalline psilocybin is crystalline psilocybin Polymorph A′. In some embodiments, the crystalline psilocybin is crystalline psilocybin Polymorph B.

In some embodiments, the psilocybin comprises amorphous psilocybin.

### Pharmaceutically Acceptable Excipients:

In some embodiments, the composition comprises at least one pharmaceutically acceptable excipient.

In some embodiments, the composition comprises at least one pharmaceutically acceptable excipient that does not substantially chemically or physically interact with a functional group of psilocybin. In some embodiments, the composition comprises at least one pharmaceutically acceptable excipient that does not substantially chemically or physically interact with the phosphonate ester of psilocybin. In some embodiments, the composition comprises at least one pharmaceutically acceptable excipient that does not form a covalent bond and/or hydrogen bond with the phosphonate ester of psilocybin.

In some embodiments, the composition does not contain a pharmaceutically acceptable excipient that substantially chemically or physically interacts with a functional group of psilocybin. In some embodiments, the composition does not contain a pharmaceutically acceptable excipient that substantially chemically or physically interacts with the phosphonate ester of psilocybin. In some embodiments, the composition does not contain a pharmaceutically acceptable excipient that substantially forms a covalent bond and/or hydrogen bond with the phosphonate ester of psilocybin. In some embodiments, the composition does not contain an inorganic silicate, silicon dioxide, or silicified excipient. In some embodiments, the composition does not contain aluminum calcium silicate, calcium silicate, diatomaceous earth, magnesium silicate, Perlite, potassium silicate, silica aerogel, silicon dioxide, sodium aluminosilicate, sodium calcium aluminosilicate, sodium silicate, talc (basic magnesium silicate) or tricalcium silicate. In some embodiments, the composition does not contain silicified microcrystalline cellulose.

In some embodiments, the composition does not contain a poly-hydroxylated excipient, for example a sugar alcohol or saccharide. In some embodiments, the composition does not contain polyols, mannitol, xylitol, sucrose, sorbitol or fructose.

In some embodiments, the composition does not contain a calcium base. In some embodiments, the composition does not contain calcium carbonate or calcium phosphate.

As noted above, the compositions of the present disclosure comprise one or more pharmaceutically acceptable excipients.

### Diluents:

In some embodiments, the composition comprises a diluent. In some embodiments, the diluent is a starch-based material.

In some embodiments, the diluent is pregelatinized starch. As referred to herein, the term “pregelatinized starch” refers to starch that that is fully pregelatinized or partially pregelatinized. In some embodiments, the pregelatinized starch is partially pregelatinized.

In some embodiments, the pregelatinized starch comprises native starch and pre-compacted starch powder. In some embodiments, the pregelatinized starch is produced by the process comprising the steps of fluidizing a mixture comprising native starch and pre-compacted starch powder; and spraying a slurry comprising pre-compacted starch powder onto the fluidized mixture. Pre-compacted starch powder is also characterized as a partially pre-gelatinized starch. However, this is distinguished from fully or completely pre-gelatinized starch, since a significant amount of intact native starch remains in the pre-compacted starch powder.

The term “native starch” refers to the starch that is derived directly from any one of a number of plant sources. Native starch is extracted from plant sources (e.g., maize), with its granule structure remaining substantially intact. The term “pre-compacted starch powder” refers to the product obtained by subjecting a non-gelatinized, granular, native starch to physical compaction with the possible input of thermal energy. After the compaction, the starch is ground and sieved to yield a free-flowing powder.

In some embodiments, the pregelatinized starch is derived from maize starch, potato starch, wheat starch or combinations thereof. In some embodiments, the pregelatinized starch is derived from maize starch. In some embodiments, the pregelatinized starch is derived from potato starch. In some embodiments, the pregelatinized starch is derived from wheat starch.

In some embodiments, the diluent is Colorcon Starcap®, Roquette Lycatab® C, Roquette Lycatab® PGS, Grain Processing Corporation (GPC) Spress® B820, Cargil C#PharmGel™, Insta-starch, Seppic Sepistab™ ST 200, or DFE Pharma Prejel PAH P5. Starcap® is a partially pregelatinized maize starch with engineered physical properties. Starcap® may be described on the basis of its physical characteristics, such as a Carr's Index of about 21, a flow rate of about 5.6 g/sec and an average mean particle size of about 90 microns. In some embodiments, the compositions of the present disclosure comprise Starcap®. Starcap® is described in U.S. Pat. No. 7,186,293 and E.P. U.S. Pat. No. 1,861,427, which are hereby incorporated by reference in their entireties for all purposes.

In some embodiments, the compositions of the present disclosure comprise a diluent characterized by its average mean particle size. In some embodiments, the average mean particle size of the diluent is about 40 to about 200 microns, e.g., about 40 microns, about 45 microns, about 50 microns, about 55 microns, about 60 microns, about 65 microns, about 70 microns, about 75 microns, about 80 microns, about 85 microns, about 90 microns, about 95 microns, about 100 microns, about 105 microns, about 110 microns, about 115 microns, about 120 microns, about 125 microns, about 130 microns, about 135 microns, about 140 microns, about 145 microns, about 150 microns, about 155 microns, about 160 microns, about 165 microns, about 170 microns, about 175 microns, about 180 microns, about 185 microns, about 190 microns, about 195 microns, or about 200 microns, including all ranges and values in between. In some embodiments, the average mean particle size of the diluent is about 50 to about 120 microns. In some embodiments, the average mean particle size of the diluent is about 60 to about 120 microns. In some embodiments, the average mean particle size of the diluent is about 90 microns. In embodiments, the average mean particle size of the diluent is about 100 microns. In embodiments, the average mean particle size of the diluent is about 150 microns.

In some embodiments, the compositions of the present disclosure comprise a diluent characterized by its flow properties, such as Carr's index, Hausner ratio or flow rate.

In some embodiments, the Carr's index of the diluent is about 12 to about 25, e.g., about 12, about 13, about 14, about 15, about 16, about 17, about 18, about 19, about 20, about 21, about 22, about 23, or about 24, including all ranges and values in between. In some embodiments, the Carr's index of the diluent is less than about 11. In some embodiments, the Carr's index of the diluent is about 12 to about 16. In some embodiments, the Carr's index of the diluent is about 18 to about 21. In some embodiments, the Carr's index of the diluent is less than about 24. In some embodiments, the Carr's index of the diluent is about 16. In some embodiments, the Carr's index of the diluent is about 17. In some embodiments, the Carr's index of the diluent is about 20. In some embodiments, the Carr's index of the diluent is about 21.

In some embodiments, the Hausner ratio of the diluent is about 1.0 to about 1.6, e.g., about 1.0, about 1.05, about 1.10, about 1.15, about 1.20, about 1.25, about 1.30, about 1.35, about 1.40, about 1.45, about 1.50, about 1.55, or about 1.60, including all ranges and values in between. In some embodiments, the Hausner ratio of the diluent is less than about 1.6. In some embodiments, the Hausner ratio of the diluent is less than about 1.2. In some embodiments, the Hausner ratio of the diluent is 1.29.

In some embodiments, the flow rate of the diluent is about 2 g/sec to about 10 g/sec, as measured using a Sotax FT300 Flowability Tester (vibration setting at 1.7 g), e.g. about 2.00 g/sec, about 2.25 g/sec, about 2.50 g/sec, about 2.75 g/sec, about 3.00 g/sec, about 3.25 g/sec, about 3.50 g/sec, about 3.75 g/sec, about 4.00 g/sec, about 4.25 g/sec, about 4.50 g/sec, about 4.75 g/sec, about 5.00 g/sec, about 5.25 g/sec, about 5.50 g/sec, about 5.75 g/sec, about 6.00 g/sec, about 6.25 g/sec, about 6.50 g/sec, about 6.75 g/sec, about 7.00 g/sec, about 7.25 g/sec, about 7.50 g/sec, about 7.75 g/sec, about 8.00 g/sec, about 8.25 g/sec, about 8.50 g/sec, about 8.75 g/sec, about 9.00 g/sec, about 9.25 g/sec, about 9.50 g/sec, about 9.75 g/sec, or about 10.00 g/sec, including all ranges and values in between. In some embodiments, the flow rate of the diluent is about 3.6 g/sec. In some embodiments, the flow rate of the diluent is about 5.6 g/sec. In some embodiments, the flow rate of the diluent is about 8.5 g/sec.

In some embodiments, the compositions of the present disclosure comprise a diluent characterized by its bulk density. In some embodiments, the bulk density of the diluent is about 0.40 g/mL to about 0.70 g/mL, e.g. about 0.40 g/mL, about 0.41 g/mL, about 0.42 g/mL, about 0.43 g/mL, about 0.44 g/mL, about 0.45 g/mL, about 0.46 g/mL, about 0.47 g/mL, about 0.48 g/mL, about 0.49 g/mL, about 0.50 g/mL, about 0.51 g/mL, about 0.52 g/mL, about 0.53 g/mL, about 0.54 g/mL, about 0.55 g/mL, about 0.56 g/mL, about 0.57 g/mL, about 0.58 g/mL, about 0.59 g/mL, about 0.60 g/mL, about 0.61 g/mL, about 0.62 g/mL, about 0.63 g/mL, about 0.64 g/mL, about 0.65 g/mL, about 0.66 g/mL, about 0.67 g/mL, about 0.68 g/mL, about 0.69 g/mL, or about 0.70 g/mL, including all ranges and values in between. In some embodiments, the bulk density of the diluent is about 0.47 g/mL. In some embodiments, the bulk density of the diluent is about 0.63 g/mL.

In some embodiments, the compositions of the present disclosure comprise a diluent characterized by its moisture content. In some embodiments, the moisture content of the diluent is about 1% to about 20%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, including all ranges and values in between. In some embodiments, the moisture content of the diluent is about 6%. In some embodiments, the moisture content of the diluent is less than about 15%.

In some embodiments, the composition comprises about 85-99% by weight of a diluent, e.g., about 85%, about 86%, about 87%, about 88%, about 89%, about 90%, about 91%, about 92%, about 93%, about 94%, about 95%, about 96%, about 97%, about 98%, or about 99%, including all ranges and values therebetween.

In some embodiments, the diluent is pregelatinized starch. In some embodiments, the composition comprises about 85% to about 99% pregelatinized starch, e.g., about 85%, about 86%, about 87%, about 88%, about 89%, about 90%, about 91%, about 92%, about 93%, about 94%, about 95%, about 96%, about 97%, about 98%, or about 99%, including all ranges and values in between. In some embodiments, the composition comprises about 98% pregelatinized starch. In some embodiments, the composition comprises about 94% pregelatinized starch. In some embodiments, the composition comprises about 89% pregelatinized starch.

### Lubricants:

In some embodiments, the composition comprises a lubricant. In some embodiments, the lubricant is selected from the group consisting of sodium stearyl fumarate, magnesium stearate, calcium stearate, zinc stearate, glyceryl monostearate, glyceryl behenate, glyceryl palmostearate, magnesium lauryl stearate, stearic acid, talc, and hydrogenated castor oil. In some embodiments, the lubricant is sodium stearyl fumarate.

In some embodiments, the composition comprises about 0.5-2.0% by weight of a lubricant, e.g., about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%, including all ranges and values in between.

In some embodiments, the lubricant is sodium stearyl fumarate. In some embodiments, the composition comprises about 0.5% to about 2.0% sodium stearyl fumarate, e.g., about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%, including all ranges and values in between. In some embodiments, the composition comprises about 1% sodium stearyl fumarate.

In some embodiments, the composition comprises about 0.5-2% by weight of the lubricant, e.g., about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween.

### Glidants:

In some embodiments, the composition comprises a glidant. In some embodiments, the glidant is selected from the group consisting of calcium phosphate trihydrate, calcium silicate, cellulose, powdered, colloidal silicon dioxide, magnesium silicate, silicon dioxide, starch, talc, and combinations thereof.

In some embodiments, the glidant is calcium silicate. In some embodiments, the composition comprises about 0.5% to about 2% calcium silicate, e.g., about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%, including all ranges and values in between. In some embodiments, the composition comprises about 1% calcium silicate.

In some embodiments, the glidant is silicon dioxide. In some embodiments, the glidant is colloidal silicon dioxide. In some embodiments, the composition comprises about 0.1% to about 0.5% silicon dioxide, e.g., about 0.1%, about 0.15%, about 0.2%, about 0.25%, about 0.3%, about 0.35%, about 0.4%, about 0.45%, or about 0.5%, including all ranges and values in between.

In some embodiments, the glidant is magnesium silicate. In some embodiments, the composition comprises about 0.5% to about 2% magnesium silicate, e.g., about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%, including all ranges and values in between. In some embodiments, the composition comprises about 1% magnesium silicate.

In some embodiments, the glidant is talc. In some embodiments, the composition comprises about 1% to about 10% talc, e.g., about 1.0%, about 2.0%, about 3.0%, about 4.0%, about 5.0%, about 6.0%, about 7.0%, about 8.0%, about 9.0%, or about 10.0%, including all ranges and values in between.

### Disintegrants:

In some embodiments, the composition comprises a disintegrant. In some embodiments, the disintegrant is selected from the group consisting of starch (e.g., pregelatinized starch), sodium starch glycolate, povidone, crospovidone, croscarmellose sodium, polcarin, methylcellulose, hydroxypropyl cellulose (HPC), microcrystalline cellulose, carboxymethyl cellulose, sodium alginate, calcium alginate, magnesium aluminium silicate, cellulose, chitosan, colloidal silicon dioxide (SiO₂), docusate sodium, guar gum and combinations thereof. In some embodiments, the composition comprises about 0.1% to about 98% disintegrant, e.g. about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.4%, about 2.6%, about 2.8%, about 3.0%, about 3.2%, about 3.4%, about 3.6%, about 3.8%, about 4.0%, about 4.2%, about 4.4%, about 4.6%, about 4.8%, about 5.0%, about 5.5%, about 6.0%, about 6.5%, about 7.0%, about 7.5%, about 8.0%, about 8.5%, about 9.0%, about 9.5%, about 10.0%, about 11.0%, about 12.0%, about 13.0%, about 14.0%, about 15.0%, about 16.0%, about 17.0%, about 18.0%, about 19.0%, about 20.0%, about 22.0%, about 24.0%, about 26.0%, about 28.0%, about 30.0%, about 33.0%, about 36.0%, about 39.0%, about 42.0%, about 46.0%, about 50.0%, about 54.0%, about 58.0%, about 62.0%, about 68.0%, about 74.0%, about 80.0%, about 86.0%, about 92.0%, or about 98.0%, including all ranges and values in between.

In some embodiments, the disintegrant is starch. In some embodiments, the starch is pregelatinized starch. In some embodiments, the composition comprises about 50% to about 98% starch, e.g., about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, or about 98%, including all ranges and values in between.

In some embodiments, the disintegrant is sodium starch glycolate. In some embodiments, the composition comprises about 2% to about 8% sodium starch glycolate, e.g., about 2.0%, about 2.5%, about 3.0%, about 3.5%, about 4.0%, about 4.5%, about 5.0%, about 5.5%, about 6.0%, about 6.5%, about 7.0%, about 7.5%, or about 8.0%, including all ranges and values in between. In some embodiments, the composition comprises about 4% sodium starch glycolate.

In some embodiments, the disintegrant is povidone. In some embodiments, the composition comprises about 0.5% to about 5% povidone, about 0.5%, about 1.0%, about 1.5%, about 2.0%, about 2.5%, about 3.0%, about 3.5%, about 4.0%, about 4.5%, or about 5.0%, including all ranges and values in between.

In some embodiments, the disintegrant is crospovidone. In some embodiments, the composition comprises about 2% to about 5% crospovidone, e.g., about 2.0%, about 2.5%, about 3.0%, about 3.5%, about 4.0%, about 4.5%, or about 5.0%, including all ranges and values in between.

In some embodiments, the disintegrant is croscarmellose sodium. In some embodiments, the composition comprises about 10% to about 25% croscarmellose sodium, e.g., about 10%, about 11%, about 12%, about 13%, about 14%, about 15%, about 16%, about 17%, about 18%, about 19%, about 20%, about 21%, about 22%, about 23%, about 24%, or about 25%, including all ranges and values in between. In some embodiments, the composition comprises about 17.5% croscarmellose sodium.

In some embodiments, the disintegrant is polcarin potassium. In some embodiments, the composition comprises about 2% to about 10% polcarin potassium, e.g., about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0%, including all ranges and values in between. In some embodiments, the composition comprises about 2% to about 3% polcarin potassium.

In some embodiments, the disintegrant is methylcellulose. In some embodiments, the composition comprises about 2% to about 10% methylcellulose, e.g., about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0%, including all ranges and values in between. In some embodiments, the composition comprises about 5% methylcellulose.

In some embodiments, the disintegrant is hydroxypropyl cellulose. In some embodiments, the composition comprises about 2% to about 5% hydroxypropyl cellulose, e.g., about 2.0%, about 2.5%, about 3.0%, about 3.5%, about 4.0%, about 4.5%, or about 5.0%, including all ranges and values in between. In some embodiments, the composition comprises about 2% to about 3% hydroxypropyl cellulose.

In some embodiments, the disintegrant is microcrystalline cellulose. In some embodiments, the composition comprises about 5% to about 15% microcrystalline cellulose, e.g., about 5%, about 6%, about 7%, about 8%, about 9%, about 10%, about 11%, about 12%, about 13%, about 14%, or about 15%, including all ranges and values in between. In some embodiments, the composition comprises about 10% microcrystalline cellulose.

In some embodiments, the disintegrant is carboxymethyl cellulose. In some embodiments, the composition comprises about 1% to about 6% carboxymethyl cellulose, e.g., about 1.0%, about 1.5%, about 2.0%, about 2.5%, about 3.0%, about 3.5%, about 4.0%, about 4.5%, about 5.0%, about 5.5%, or about 6.0%, including all ranges and values in between. In some embodiments, the composition comprises about 2% to about 3% carboxymethyl cellulose.

In some embodiments, the disintegrant is sodium alginate. In some embodiments, the composition comprises about 2.5% to about 10% sodium alginate, e.g., about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0%, including all ranges and values in between. In some embodiments, the composition comprises about 4% to about 5% sodium alginate.

In some embodiments, the disintegrant is calcium alginate. In some embodiments, the composition comprises about 2.5% to about 10% calcium alginate, e.g., about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0%, including all ranges and values in between. In some embodiments, the composition comprises about 4% to about 5% calcium alginate.

In some embodiments, the disintegrant is magnesium aluminum silicate

In some embodiments, the disintegrant is chitosan.

In some embodiments, the disintegrant is colloidal silicon dioxide. In some embodiments, the composition comprises about 0.1% to about 0.55% colloidal silicon dioxide, e.g., about 0.1%, about 0.15%, about 0.2%, about 0.25%, about 0.3%, about 0.35%, about 0.4%, about 0.45%, about 0.5%, or about 0.55%, including all ranges and values in between.

In some embodiments, the disintegrant is cellulose. In some embodiments, the composition comprises about 5% to about 15% cellulose, e.g., about 5%, about 6%, about 7%, about 8%, about 9%, about 10%, about 11%, about 12%, about 13%, about 14%, or about 15%, including all ranges and values in between.

In some embodiments, the disintegrant is docusate sodium. In some embodiments, the composition comprises about 0.1% to about 1.0% docusate sodium, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, In some embodiments, the composition comprises about 0.5% docusate sodium.

### Dosage Forms:

According to the present disclosure, the pharmaceutical composition may be in any pharmaceutically acceptable form that maintains the integrity and potency of the psilocybin contained therein and provides pharmaceutically acceptable psilocybin content uniformity (as measured by European Pharmacopeia 2.96, or USP <905>). In some embodiments, the pharmaceutical composition is in solid form. In some embodiments, the pharmaceutical composition is an oral dosage form. In some embodiments, the pharmaceutical composition is solution (for example, an aqueous solution). In some embodiments, the pharmaceutical composition is a tablet. In some embodiments, the pharmaceutical composition is a capsule. In some embodiments, the pharmaceutical composition is a powder.

In some embodiments, the pharmaceutical composition comprises psilocybin; about 85-99% by weight of a diluent and about 0.5-2% by weight of the lubricant.

In some embodiments, the composition comprises: psilocybin; about 85-99% by weight of a diluent, e.g., about 85%, about 86%, about 87%, about 88%, about 89%, about 90%, about 91%, about 92%, about 93%, about 94%, about 95%, about 96%, about 97%, about 98%, or about 99%, including all ranges and values therebetween; and about 0.5-2% by weight of the lubricant, e.g., about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween.

In some embodiments, the composition comprises: psilocybin; about 85-99% by weight of Pregelatinized starch (Starcap), e.g., about 85%, about 86%, about 87%, about 88%, about 89%, about 90%, about 91%, about 92%, about 93%, about 94%, about 95%, about 96%, about 97%, about 98%, or about 99%, including all ranges and values therebetween; and about 0.5-2% by weight of Sodium Stearyl Fumarate, e.g., about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween.

In some embodiments, the composition comprises about 1% by weight of psilocybin, about 98% by weight of a diluent, and about 1% by weight of a lubricant. In some embodiments, the composition comprises about 1% by weight of psilocybin, about 98% by weight of Pregelatinized starch (Starcap), and about 1% by weight of Sodium Stearyl Fumarate.

In some embodiments, the composition comprises about 5% by weight of psilocybin, about 94% by weight of a diluent, and about 1% by weight of a lubricant. In some embodiments, the composition comprises about 5% by weight of psilocybin, about 94% by weight of Pregelatinized starch (Starcap), and about 1% by weight of Sodium Stearyl Fumarate.

In some embodiments, the composition comprises about 10% by weight of psilocybin, about 89% by weight of a diluent, and about 1% by weight of a lubricant. In some embodiments, the composition comprises about 10% by weight of psilocybin, about 89% by weight of Pregelatinized starch (Starcap), and about 1% by weight of Sodium Stearyl Fumarate.

In some embodiments, the pharmaceutical composition is a capsule. In some embodiments, the capsule material comprises methylcellulose, gellan gum, carrageenan, pectin, glycerin, hydroxypropyl (HP) starch, plasticizer, buffer (e.g., phosphate buffer), amorphous starch (e.g., amylopectin), a softener or combinations thereof. In some embodiments, the capsule material comprises hydroxypropyl methylcellulose. In some embodiments, the capsule material comprises starch (e.g., hydroxypropyl starch or amylopectin). In some embodiments, the capsule material is VcapsPlus®, Vcaps®, Quali-V®, EMBROCaps®-VG, VegiCaps®, SeaGel™, or VegaGels®.

In some embodiments, the capsule material comprises about 1% to about 7% moisture content, e.g., about 1%, about 2%, about 3%, about 4%, about 5%, about 6% or about 7%, including all ranges and values in between. In some embodiments, the capsule material comprises less than about 6% moisture content. In some embodiments, the capsule material comprises about 3% to about 7% moisture content. In some embodiments, the capsule material comprises about 4% to about 6% moisture content.

In some embodiments, the content uniformity of the compositions of the present disclosure comply with the European Pharmacopeia 2.96, or USP <905>.

### Powder Blends:

In some embodiments, the compositions of the present disclosure comprise a powder blend (i.e., a mixture of psilocybin and one or more pharmaceutically acceptable excipients) that is suitable for use in a capsule. In some embodiments, the compositions of the present disclosure comprise a powder blend that is suitable for direct compression to provide a tablet.

In some embodiments, the powder blends of the present disclosure are characterized by their flow properties, such as the Carr's index or the Hausner ratio.

In some embodiments, the Carr's index of the powder blend is about 12 to about 25, e.g., about 12, about 13, about 14, about 15, about 16, about 17, about 18, about 19, about 20, about 21, about 22, about 23, or about 24 including all ranges and values in between. In some embodiments, the Carr's index of the powder blend is less than about 11. In some embodiments, the Carr's index of the powder blend is about 12 to about 16. In some embodiments, the Carr's index of the powder blend is about 18 to about 21. In some embodiments, the Carr's index of the powder blend is less than about 24. In some embodiments, the Carr's index of the powder blend is about 16. In some embodiments, the Carr's index of the powder blend is about 17. In some embodiments, the Carr's index of the powder blend is about 20. In some embodiments, the Carr's index of the powder blend is about 21.

In some embodiments, the Hausner ratio of the powder blend is about 1.0 to about 1.6, e.g., about 1.00, about 1.05, about 1.10, about 1.15, about 1.20, about 1.25, about 1.30, about 1.35, about 1.40, about 1.45, about 1.50, about 1.55, or about 1.6, including all ranges and values in between. In some embodiments, the Hausner ratio of the powder blend is less than about 1.6. In some embodiments, the Hausner ratio of the powder blend is less than about 1.2. In some embodiments, the Hausner ratio of the powder blend is 1.29.

In some embodiments, the powder blends of the present disclosure are characterized by their bulk density In some embodiments, the bulk density of the powder blend is about 0.40 g/mL to about 0.70 g/mL, e.g. about 0.40 g/mL, about 0.41 g/mL, about 0.42 g/mL, about 0.43 g/mL, about 0.44 g/mL, about 0.45 g/mL, about 0.46 g/mL, about 0.47 g/mL, about 0.48 g/mL, about 0.49 g/mL, about 0.50 g/mL, about 0.51 g/mL, about 0.52 g/mL, about 0.53 g/mL, about 0.54 g/mL, about 0.55 g/mL, about 0.56 g/mL, about 0.57 g/mL, about 0.58 g/mL, about 0.59 g/mL, about 0.60 g/mL, about 0.61 g/mL, about 0.62 g/mL, about 0.63 g/mL, about 0.64 g/mL, about 0.65 g/mL, about 0.66 g/mL, about 0.67 g/mL, about 0.68 g/mL, about 0.69 g/mL, or about 0.70 g/mL, including all ranges and values in between. In some embodiments, the bulk density of the powder blend is about 0.44 g/mL to 0.52 g/mL. In some embodiments, the bulk density of the powder blend is about 0.45 g/mL to 0.55 g/mL. In some embodiments, the bulk density of the powder blend is about 0.47 g/mL. In some embodiments, the bulk density of the composition is about 0.63 g/mL.

In some embodiments, the powder blends of the present disclosure are characterized by their ratio of native starch to pre-compacted starch. In some embodiments, the ratio of native starch to pre-compacted starch is about 70:30 to about 99:1, e.g., about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1, including all ranges and values in between. In some embodiments, ratio of native starch to pre-compacted starch is about 85:15 to about 95:5.

In some embodiments, the powder blend comprises: psilocybin; about 85-99% by weight of a diluent and about 0.5-2% by weight of the lubricant.

In some embodiments, the powder blend comprises: psilocybin about 85-99% by weight of a diluent, e.g., about 85%, about 86%, about 87%, about 88%, about 89%, about 90%, about 91%, about 92%, about 93%, about 94%, about 95%, about 96%, about 97%, about 98%, or about 99%, including all ranges and values therebetween; and about 0.5-2% by weight of the lubricant, e.g., about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween.

In some embodiments, the powder blend comprises: psilocybin; about 85-99% by weight of Pregelatinized starch (Starcap), e.g., about 85%, about 86%, about 87%, about 88%, about 89%, about 90%, about 91%, about 92%, about 93%, about 94%, about 95%, about 96%, about 97%, about 98%, or about 99%, including all ranges and values therebetween; and about 0.5-2% by weight of Sodium Stearyl Fumarate, e.g., about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween.

In some embodiments of the present method, the powder blend comprises about 1% to 10% by weight of psilocybin, e.g., about 1%, about 2%, about 3%, about 4%, about 5%, about 6%, about 7%, about 8%, about 9%, or about 10%, including all ranges and values therebetween. In some embodiments, the powder blend comprises about 1% to 15% by weight of psilocybin, e.g., about 1%, about 2%, about 3%, about 4%, about 5%, about 6%, about 7%, about 8%, about 9%, about 10%, about 11%, about 12%, about 13%, about 14% or about 15%, including all ranges and values therebetween.

In some embodiments, the powder blend comprises about 1% by weight of psilocybin, about 98% by weight of a diluent, and about 1% by weight of a lubricant. In some embodiments, the powder blend comprises about 1% by weight of psilocybin, about 98% by weight of Pregelatinized starch (Starcap), and about 1% by weight of Sodium Stearyl Fumarate.

In some embodiments, the powder blend comprises about 5% by weight of psilocybin, about 94% by weight of a diluent, and about 1% by weight of a lubricant. In some embodiments, the powder blend comprises about 5% by weight of psilocybin, about 94% by weight of Pregelatinized starch (Starcap), and about 1% by weight of Sodium Stearyl Fumarate.

In some embodiments, the composition comprises about 10% by weight of psilocybin, about 89% by weight of a diluent, and about 1% by weight of a lubricant. In some embodiments, the powder blend comprises about 10% by weight of psilocybin, about 89% by weight of Pregelatinized starch (Starcap), and about 1% by weight of Sodium Stearyl Fumarate.

In some embodiments, the content uniformity of the powder blends of the present disclosure comply with the European Pharmacopeia 2.96, or USP <905>.

### Methods of Making the Pharmaceutical Compositions of the Present Disclosure

In some embodiments, the present disclosure provides a method of making a pharmaceutical composition comprising: (a) direct mixing psilocybin and one or more pharmaceutically acceptable excipients to provide a blend and (b) filling a capsule with the blend to provide the pharmaceutical composition, wherein the content uniformity of the composition complies with the European Pharmacopeia 2.96, or USP <905>.

In some embodiments, the blend is prepared by blending psilocybin and the one or more pharmaceutically acceptable excipients for about 15 to about 60 minutes, e.g., about 15 minutes, about 20 minutes, about 25 minutes, about 30 minutes, about 35 minutes, about 40 minutes, about 45 minutes, about 50 minutes, about 55 minutes or about 60 minutes. In some embodiments, the blending is carried out in one or more stages. In some embodiments, the blending is carried out in 2 stages. In some embodiments, the blending is carried out in 3 stages.

In some embodiments of the present method, the mass balance of psilocybin and related substances in the composition decreases by less than 3% after storage of the composition at 40° C. and 75% relative humidity for one month. In some embodiments, the mass balance of psilocybin and related substances in the composition decreases by less than 3% after storage of the composition at 40° C. and 75% relative humidity for two months.

In some embodiments of the present method, the blend comprises about 1% to 10% by weight of psilocybin, e.g., about 1%, about 2%, about 3%, about 4%, about 5%, about 6%, about 7%, about 8%, about 9%, or about 10%, including all ranges and values therebetween. In some embodiments, the blend comprises about 1% to 15% by weight of psilocybin, e.g., about 1%, about 2%, about 3%, about 4%, about 5%, about 6%, about 7%, about 8%, about 9%, about 10%, about 11%, about 12%, about 13%, about 14% or about 15%, including all ranges and values therebetween. In some embodiments, the capsule contains 1 mg to 40 mg of psilocybin, e.g., 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg, including all ranges and values therebetween. In some embodiments, the capsule comprises about 1 mg to 25 mg of psilocybin, e.g., 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 22 mg, 24 mg, or 25 mg, including all ranges and values therebetween. In some embodiments, the capsule comprises about 1 mg to 10 mg of psilocybin, e.g., 1 mg, 2 mg, 3 mg, 4 mg, 5 mg, 6 mg, 7 mg, 8 mg, 9 mg, or 10 mg, including all ranges and values therebetween. In some embodiments, the capsule comprises about 1 mg to 5 mg of psilocybin, e.g., 1 mg, 2 mg, 3 mg, 4 mg, or 5 mg, including all ranges and values therebetween. In some embodiments, the capsule contains 1 mg, 5 mg, 10 mg, or 25 mg of psilocybin. In some embodiments, the capsule contains 1 mg of psilocybin. In some embodiments, the capsule contains 5 mg of psilocybin. In some embodiments, the capsule contains 10 mg of psilocybin. In some embodiments, the capsule contains 15 mg of psilocybin. In some embodiments, the capsule contains 20 mg of psilocybin. In some embodiments, the capsule contains 25 mg of psilocybin.

In some embodiments of the present method, the blend comprises one or more pharmaceutically acceptable excipients. In some embodiments, the one or more pharmaceutically acceptable excipients comprises a diluent. In some embodiments, the diluent is pregelatinized starch.

In some embodiments of the present method, the blend further comprises a lubricant. In some embodiments, the lubricant is selected from the group consisting of sodium stearyl fumarate, magnesium stearate, calcium stearate, zinc stearate, glyceryl monostearate, glyceryl behenate, glyceryl palmostearate, magnesium lauryl stearate, stearic acid, talc, and hydrogenated castor oil. In some embodiments, the lubricant is sodium stearyl fumarate.

In some embodiments, the blend does not contain a glidant.

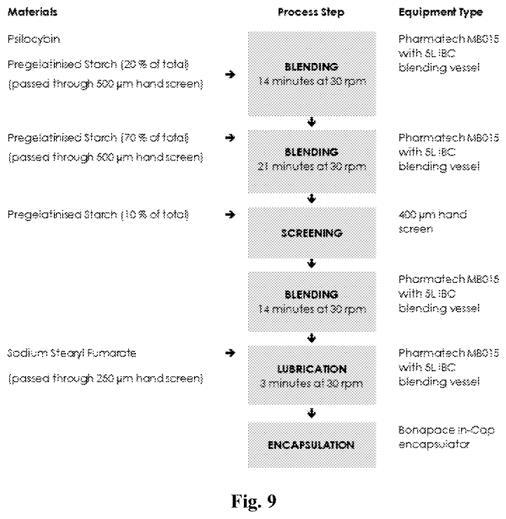

In some embodiments, the method of making a pharmaceutical composition of the present disclosure is the method described in FIG. 9.

Without being bound by any particular theory, it was surprisingly found that the disclosed methods of making pharmaceutical compositions by direct mixing psilocybin and one or more pharmaceutically acceptable excipients provide compositions with pharmaceutically acceptable content uniformity (as measured by European Pharmacopeia 2.96, or USP <905>) even at low levels of drug loading.

### Methods of Treating Depressive Disorders

In some embodiments, the methods provided herein are used to treat a subject with a depressive disorder, comprising administering a therapeutically effect of a composition disclosed herein. In some embodiments, the methods provided herein are used to treat a subject with a mood disorder, comprising administering a therapeutically effect of a composition disclosed herein. The compositions of the present disclosure may be used to treat any condition or disorder that is treatable by administration of psilocybin. Methods of the present disclosure may be used to treat disorders described in PCT Publication No. WO2020/212951, WO2020/212948 and WO2020/212952, which are hereby incorporated by reference in their entireties for all purposes.

In some embodiments, the depressive disorder is major depressive disorder, atypical depression, bipolar disorder, catatonic depression, depressive disorder due to a medical condition, postpartum depression, premenstrual dysphoric disorder, or seasonal affective disorder.

In some embodiments, a depressive disorder comprises a medical diagnosis based on the criteria and classification from Diagnostic and Statistical Manual of Medical Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with “treatment-resistant depression”. The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, or 5 treatment attempts. In some embodiments, the subject with treatment-resistant depression has been diagnosed with major depressive disorder and has failed to respond to 3 or more treatment attempts. In some embodiments, the subject with treatment-resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, no other treatment is administered to the subject to reduce the sign or symptom of depression after administration of the psilocybin.

In some embodiments, the method of the present disclosure further comprises administering to the subject at least one additional therapeutic to reduce the sign or symptom of depression. In some embodiments, at least one additional therapeutic is a selective serotonin reuptake inhibitor, a serotonin and norepinephrine reuptake inhibitor, a tricyclic antidepressant, a tetracyclic antidepressant, a dopamine reuptake inhibitor, a 5-HT1A receptor antagonist, a 5-HT2 receptor antagonist, a 5-HT3 receptor antagonist, a monoamine oxidase inhibitor, or a noradrenergic antagonist. In some embodiments, at least one additional therapeutic is administered prior to administration of psilocybin, on the same day as the administration of psilocybin, or after administration of psilocybin.

In some embodiments, the subject with the depressive disorder has an additional comorbidity or disorder. In some embodiments, the additional comorbidity or disorder is an anxiety disorder, an obsessive-compulsive disorder, alcoholism, a personality disorder, a cardiovascular disease, a neurological disease, or cancer. In some embodiments, the subject has dementia, Alzheimer's Disease, or Parkinson's Disease. In some embodiments, reducing at least one sign or symptom of depression in the subject using the methods of the present disclosure prevents one or more comorbidities or disorders in the subject.

### EXAMPLES

The following examples, which are included herein for illustration purposes only, are not intended to be limiting.

### Example 1: Composition and Stability of Psilocybin Tablets Containing Silicified Microcrystalline Starch

Following formulation optimization, 1 mg and 5 mg psilocybin-containing tablets having the shown in Table 8 were prepared and subjected to 6-month drug product stability studies.

TABLE 8
Unit Formula for 1 mg and 5 mg Psilocybin Tablets

| Excipient | 1 mg (%) | 1 mg (g) | 5 mg (%) | 5 mg (g) |
|---|---|---|---|---|
| Psilocybin | 1.0 | 7.0 | 5.0 | 35.0 |
| (COM 360) | | | | |
| Silicified | 20.5 | 143.5 | 12.5 | 87.5 |
| Microcrystalline | | | | |
| Cellulose | | | | |
| (SMCC 50) | | | | |
| Silicified | 75.5 | 528.5 | 79.5 | 556.5 |
| Microcrystalline | | | | |
| Cellulose | | | | |
| (SMCC 90) | | | | |
| Sodium Starch | 1.0 | 7.0 | 1.0 | 7.0 |
| Glycolate | | | | |
| Colloidal silicon | 1.0 | 7.0 | 1.0 | 7.0 |
| Dioxide (Aerosil) | | | | |
| Sodium Steary1 | 1.0 | 7.0 | 1.0 | 7.0 |
| Fumarate (Pruv) | | | | |
| TOTAL | 100.0 | 700.0 | 100.0 | 700.0 |

Results: The stability study storage conditions and results are shown in Table 9 and Table 10, below. Upon storage, the tablets became discolored and lost API potency. However, there was no obvious, commensurate increase in psilocybin-related impurities, for example, psilocin (i.e., there was an apparent loss of mass balance). Thus, even when the tablets were stored in the presence of a desiccant, the drug product stability was determined to be unsuitable for a pharmaceutical product.

TABLE 9
Stability of 1 mg Psilocybin Tablet

| Storage | | | | | |
|---|---|---|---|---|---|
| condition | Initials | T = 1 m | T = 3 m | T = 6 m | Comments |
| 25′C./60% RH | 96.0% | 93.0% | 95.0% | 92.0% | No mass balance |
| (with | | | | | and no obvious |
| desiccant) | | | | | linear increases in |
| | | | | | psilocin |
| 40′C./75% RH | 96.0% | 92.0% | 93.0% | 93.0% | No mass balance |
| (with | | | | | and no obvious |
| desiccant) | | | | | linear increases in |
| | | | | | psilocin |
| 40′C./75% RH | 96.0% | 93.0% | 91.0% | 81.0% | No mass balance |
| (without | | | | | and no obvious |
| desiccant) | | | | | linear increases in |
| | | | | | psilocin. |

TABLE 10
Stability of Psilocybin 5 mg Tablets

| Storage | | | | | |
|---|---|---|---|---|---|
| condition | Initials | T = 1 m | T = 3 m | T = 6 m | Comments |
| 25′C./60% RH | 100.0% | 98.0% | 97.0%¹ | 94.0% | No mass balance |
| (with | | | | | and no obvious |
| desiccant) | | | | | linear increases in |
| | | | | | psilocin |
| 40′C./75% RH | 100.0% | 98.0% | 98.0%¹ | 96.0% | No mass balance |
| (with | | | | | and no obvious |
| desiccant) | | | | | linear increases in |
| | | | | | psilocin |
| 40′C./75% RH | 100.0% | 98.0% | 96.0%¹ | 93.0% | No mass balance |
| (without | | | | | and no obvious |
| desiccant) | | | | | linear increases in |
| | | | | | psilocin |

¹browning first observed

Discussion: To determine the role that water may play in the loss of mass balance, a moisture barrier aqueous film coating was applied to the tablet to reduce moisture penetration. However, the coated tablets had even lower stability. Another possibility is that the amorphous form could have been formed during compression of the tablet. Amorphous forms are less stable than their crystalline counterparts, and in certain circumstances degrade via different mechanisms, which could potentially explain the lack of obvious API degradation. This too was ruled out after it became clear that low recoveries were being seen when blends were manufactured by other methods, again without accompanying degradation. Furthermore, similarly instability was observed in capsules that were prepared by filling empty capsules with the tablet formulation blend (i.e., the excipient blend of Table 8).

### Example 2: Composition and Stability of Psilocybin Capsules in the Absence of Silicon

### Dioxide-Based Excipients

Experiments were conducted to explore whether the observed loss of mass balance upon product storage resulted from the interaction of silanized microcrystalline cellulose and/or colloidal silica (both added to enhance flow of the API) with the psilocybin (e.g., irreversible binding of the psilocybin to the excipient surface).

Without being bound by any theory, it was hypothesized that psilocybin (via its phosphonate ester group) could form a strong interaction (e.g., H-bond) with the silanized surface resulting in a loss of potency without a commensurate increase in psilocybin-related impurities.

Two capsule formulations using starch-based excipients starch 1500 and Starcap were evaluated. Blends containing 1% (1 mg) and 10% (25 mg) of psilocybin were prepared with each excipient. Table 11 shows the physical properties of each blend. Starcap showed superior filling and content uniformity as shown in.

TABLE 11
Comparison of Starch 1500 and Starcap Formulations

| Batch Details | Starch 1500 | | Starcap | |
|---|---|---|---|---|
| Batch size (g) | 700 | 700 | 700 | 100 |
| Blend Strength | 1% | 10% | 1% | 10% |
| Capsule Strength (mg) | 1 | 25 | 25 | 1 |
| Bulk Density (g/ml) | 0.66 | 0.56 | 0.50 | 048 |
| Carr's Index (%) | 17 | 21 | 16 | 20 |
| Mean Blend Assay | 101 | 101 | 98 | 98 |
| Blend RSD (%) | 1.9 | 0.7 | 3.8 | 1.1 |
| Mean capsule fill weight (mg) | 112 | 243 | 100 | 259 |
| range | 109-117 | 219-269 | 94-105 | 252-269 |
| Fill Weight RSD (%) | 2.1 | 6.1* | 2.7 | 1.6 |
| Mean Capsule Assay 9%) | 99 | 96 | 97 | 98 |
| Mean content uniformity (%) | 99 | 97 | 98 | 96 |
| Content Uniformity, AV | 4.3 | 19.8* | 5.5 | 9.0 |

*Fail

The Starcap formulation was selected for further development. Table 12 shows the capsule composition and FIG. 9 shows the method of preparing the composition using a direct mixing method. These capsules were tested for their stability, below.

TABLE 12
Unit Formula for Psilocybin Capsules

| Excipient | 1 mg (%) | 1 mg (g) | 5 mg (%) | 5 mg (g) | 25 mg (%) | 25 mg (g) |
|---|---|---|---|---|---|---|
| Psilocybin (COM | 1.0 | 7.0 | 5.0 | 35.0 | 10.0 | 70.0 |
| 360) | | | | | | |
| Pregelatinized | 98.0 | 686 | 94.0 | 658 | 89.0 | 623.0 |
| starch (Starcap) | | | | | | |
| Sodium Stearyl | 1.0 | 7.0 | 1.0 | 7.0 | 1.0 | 7.0 |
| Fumarate (Pruv) | | | | | | |
| Capsule, HPMC, | 1 unit | Size 2* | 1 unit | Size 2* | 1 unit | Size 0** |
| white opaque | | | | | | |
| TOTAL | 100.0 | 700.0 | 100.0 | 700.0 | 100.0 | 700.0 |

*fill weight 100 mg,
**fill weight 250mg

Table 13 shows the results from storage of the 1 mg capsules (with desiccant) at 25° C./60% RH for one and two months. The data shows that the capsules were within specification and complied with all measured parameters at both timepoints.

TABLE 13
Stability Data for Psilocybin 1 mg Capsules
(with Desiccant)-Stored at 25° C./60% RH

| Attribute | Method | Specification | Initial | 1M | 2M |
|---|---|---|---|---|---|
| Appearance | Visual | Off-white powder in a | Complies | Complies | Complies |
| | | white size 2 capsule | | | |
| Assay | HPLC | 90.0%-110.0% LC | 100.5% | 100.6% | 98.8% |
| | | (0.90-1.10 mg) | | | |
| Related | HPLC | Psilocin NMT 1.0% | 0.5% | 0.4% | 0.4% |
| Substances | | Ind. impurities | | | |
| | | NMT 0.5% | | | |
| | | RRT 1.24 | 0.1% | 0.1% | 0.1% |
| | | RRT 1.35 | ND | ND | 0.1% |
| | | RRT 1.51 | ND | 0.1% | ND |
| | | Total Impurities | 0.5% | 0.6% | 0.6% |
| | | NMT 3.0% | | | |
| Moisture | Ph. Eur. | Record Results | 7.66% | 3.05% | 4.80% |
| content | 2.2.32 | | | | |
| Dissolution | Ph. Eur. | Q = 80% at 45 mins | 98% | 102% | 105% |
| | 2.9.3 | | | | |
| | Paddle/HPLC | | | | |

Table 14 shows the results from storage of the 1 mg capsules (with desiccant) at 40° C./75% RH for one and two months. The data shows that the capsules were within specification and complied with all measured parameters at both timepoints.

TABLE 14
Stability Data for Psilocybin 1 mg Capsules
(with Desiccant)-Stored at 40° C./75% RH

| Attribute | Method | Specification | Initial | 1M | 2M |
|---|---|---|---|---|---|
| Appearance | Visual | Off-white powder in a | Complies | Complies | Complies |
| | | white size 2 capsule | | | |
| Assay | HPLC | 90.0%-110.0% of label | 100.5% | 101.8% | 100.1% |
| | | claim | | | |
| | | (0.90-1.10 mg) | | | |
| Related | HPLC | Psilocin NMT 1.0% | 0.5% | 0.4% | 0.4% |
| substances | | Individual imps | | | |
| | | NMT 0.5% | | | |
| | | RRT 0.85 | ND | 0.1% | ND |
| | | RRT 1.25 | 0.1% | 0.1% | 0.1% |
| | | RRT 1.38 | ND | 0.1% | 0.1% |
| | | RRT 1.44 | ND | 0.1% | ND |
| | | RRT 1.51 | ND | 0.1% | ND |
| | | Total impurities | 0.5% | 0.8% | 0.5% |
| | | NMT 3.0% | | | |
| Moisture | Ph. Eur. | Record Results | 7.66% | 4.07% | 5.60% |
| content | 2.2.32 | | | | |
| Dissolution | Ph. Eur. | Q = 80% at 45 mins | 98% | 102% | 104% |
| | 2.9.3 | | | | |
| | Paddle/HPLC | | | | |

Table 15 shows the results from storage of the 5 mg capsules (with desiccant) at 25° C./60% RH for one and two months. The data shows that the capsules were within specification and complied with all measured parameters at both timepoints.

TABLE 15
Stability Data for Psilocybin 5 mg Capsules (with Desiccant)-Stored at 25° C./60% RH

| Attribute | Method | Specification | Initial | 1M | 2M |
|---|---|---|---|---|---|
| Appearance | Visual | Off-white powder in a | Complies | Complies | Complies |
| | | white size 2 capsule | | | |
| Assay | HPLC | 90.0%-110.0% of LC | 99.7% | 99.5% | 99.2% |
| | | (4.50-5.50 mg) | | | |
| Related | HPLC | Psilocin NMT 1.0% | 0.5% | 0.5% | 0.4% |
| substances | | Individual imps NMT 0.5% | | | |
| | | RRT 1.26 | 0.1% | 0.1% | 0.1% |
| | | RRT 1.39 | ND | ND | 0.1% |
| | | Total impurities NMT 3.0% | 0.5% | 0.6% | 0.5% |
| Moisture | Ph Eur | Record Results | 10.71% | 6.31% | 6.26% |
| content | 2.2.32 | | | | |
| Dissolution | Ph. Eur. | Q = 80% at 45 mins | 99% | 99% | 102% |
| | 2.9.3 | | | | |
| | Paddle/HPLC | | | | |

Table 16 shows the results from storage of the 5 mg capsules (with desiccant) at 40° C./75% RH for one and two months. The data shows that the capsules were within specification and complied with all measured parameters at both timepoints.

TABLE 16
Stability Data for Psilocybin 5 mg Capsules (with Desiccant)-Stored at 40° C./75% RH

| Attribute | Method | Specification | Initial | 1M | 2M |
|---|---|---|---|---|---|
| Appearance | Visual | Off-white powder in a white | Complies | Complies | Complies |
| | | size 2 capsule | | | |
| Assay | HPLC | 90.0%-110.0% of LC | 99.7% | 100.0% | 98.4% |
| | | (4.50-5.50 mg) | | | |
| Related | HPLC | Psilocin NMT 1.0% | 0.5% | 0.5% | 0.4% |
| substances | | Individual imps NMT 0.5% | | | |
| | | RRT 1.24 | 0.1% | 0.1% | 0.1% |
| | | RRT 1.37 | ND | 0.1% | 0.1% |
| | | Total Impurities NMT 3.0% | 0.5% | 0.7% | 0.5% |
| Moisture | Ph. Eur. | Record Results | 10.71% | 5.94% | 6.58% |
| Content | 2.2.32 | | | | |
| Dissolution | Ph. Eur. | Q = 80% at 45 mins | 99% | 98% | 101% |
| | 2.9.3 | | | | |
| | Paddle/HPLC | | | | |

Table 17 shows the results from storage of the 25 mg capsules (with desiccant) at 25° C./60% RH for one and two months. The data shows that the capsules were within specification and complied with all measured parameters at both timepoints.

TABLE 17
Stability Data for Psilocybin 25 mg Capsules (with Desiccant) - Stored at 25° C./60% RH

| Attribute | Method | Specification | | Initial | 1M | 2M |
|---|---|---|---|---|---|---|
| Appearance | Visual | Off-white powder in a | | Complies | Complies | Complies |
| | | white size 0 capsule | | | | |
| Assay | HPLC | 90.0%-110.0% of LC | | 101.0% | 99.8% | 99.4% |
| | | (22.5-27.50 mg) | | | | |
| Related | HPLC | Psilocin NMT | 1.0% | 0.5% | 0.4% | 0.4% |
| substances | | Individual imps NMT | 0.5% | | | |
| | | RRT | 1.26 | 0.1% | 0.1% | 0.1% |
| | | RRT | 1.37 | ND | 0.1% | 0.1% |
| | | Total Impurities NMT | 3.0% | 0.5% | 0.6% | 0.5% |

| | | | | | |
|---|---|---|---|---|---|
| Moisture | Ph. Eur. | Record Results | 8.29% | 7.64% | 7.39% |
| Content | 2.2.32 | | | | |
| Dissolution | Ph. Eur. | Q = 80% at 45 mins | 100% | 101% | 102% |
| | 2.9.3 | | | | |
| | Paddle/ | | | | |
| | HPLC | | | | |

Table 18 shows the results from storage of the 25 mg capsules (with desiccant) at 40° C./75% RH for one and two months. The data shows that the capsules were within specification and complied with all measured parameters at both timepoints.

The HPLC data referred to in this Example were obtained using the Related Substance HPLC Method described herein.

TABLE 18
Stability Data for Psilocybin 25 mg Capsules (with Desiccant) - Stored at 40° C./75% RH

| Attribute | Method | Specification | | Initial | 1M | 2M |
|---|---|---|---|---|---|---|
| Appearance | Visual | Off-white powder in a | | Complies | Complies | Complies |
| | | white size 0 capsule | | | | |
| Assay | HPLC | 90.0%-110.0% of LC | | 101.3% | 99.2% | 100.4% |
| | | (22.5-27.50 mg) | | | | |
| Related | HPLC | Psilocin NMT | 1.0% | 0.5% | 0.5% | 0.4% |
| substances | | Individual imps NMT | 0.5% | | | |
| | | RRT | 1.25 | 0.1% | 0.1% | 0.1% |
| | | RRT | 1.37 | ND | 0.1% | 0.1% |
| | | Total Impurities NMT | 3.0% | 0.6% | 0.6% | 0.5% |

| | | | | | |
|---|---|---|---|---|---|
| Moisture | Ph. Eur. | Record Results | 8.29% | 7.39% | 6.64% |
| Content | 2.2.32 | | | | |
| Dissolution | Ph. Eur. | Q = 80% at 45 mins | 100% | 102% | |
| | 2.9.3 | | | | |
| | Paddle/ | | | | |
| | HPLC | | | | |

### Example 3: Composition and Stability of Psilocybin Capsules Formulated With Silicon Dioxide-Based and Other Excipients

Compositions of psilocybin with the silicon dioxide-containing excipients in Table 19 will be studied under stability conditions that are similar to those used in Example 2 and stability parameters recorded for each psilocybin composition.

TABLE 19
Silicon dioxide-based excipients
for use in stability testing.
Name
Aluminum calcium silicate
Calcium silicate
Diatomaceous earth (filter aid)
Magnesium silicate
Perlite (filter aid)
Potassium silicate
Silica aerogel
Silicon dioxides
Sodium aluminosilicate
Sodium calcium aluminosilicate
Sodium silicate
Talc (basic magnesium silicate)
Tricalcium silicate

Compositions of psilocybin with the hydrogen-bond-forming sugars or polyols in Table 20 will be studied under stability conditions that are similar to those used in Example 2 and stability parameters recorded for each psilocybin composition.

TABLE 20
Polyol and sugar alcohol excipients
for use in stability testing.

| Index | Name |
|---|---|
| 1 | Polyols |
| 2 | Mannitol |
| 3 | Xylitol |
| 4 | Sucrose |
| 5 | Sorbitol |
| 6 | Fructose |

Compositions of psilocybin with the basic excipients in Table 21 will be studied under stability conditions that are similar to those used in Example 2 and stability parameters recorded for each psilocybin composition.

TABLE 21
Basic excipients for use in stability testing.

| Index | Name |
|---|---|
| 1 | Calcium Carbonate |
| 2 | Calcium Phosphate |

### Example 4: Composition and Stability of Psilocybin Capsules Formulated with

Silicon Dioxide-Based Excipients, Pregelatinized Starch, or Combinations Thereof

A loss of potency was observed in tablet and capsules prepared from the same blend composition, as described in Examples 1 and 2. The blend contained silicified microcrystalline cellulose (SMCC) and Colloidal Silicon Dioxide (Aerosil).

Compositions of psilocybin with the silicon dioxide-containing excipients and pregelatinized starch in Table 22 were studied under stability conditions like those used in Example 2. The stability results were recorded for each psilocybin composition.

Table 22 shows the composition prepared that contains 10% by weight of silicified microcrystalline cellulose (SMCC), 1% by weight of colloidal silicon dioxide (Aerosil) and 1% by weight of psilocybin.

TABLE 22
Unit Formula for Low Level SMCC Composition.

| Excipient | Amount (%) | Mass (g) |
|---|---|---|
| Psilocybin | 1.0 | 0.7 |
| Silicified Microcrystalline | 2.5 | 1.8 |
| Cellulose (SMCC 50) | | |
| Silicified Microcrystalline | 7.5 | 5.2 |
| Cellulose (SMCC 90) | | |
| Sodium Starch Glycolate | 1.0 | 0.7 |
| Pregelatinized Starch (StarCap) | 86.0 | 60.2 |
| Colloidal silicon Dioxide (Aerosil) | 1.0 | 0.7 |
| Sodium Stearyl Fumarate (Pruv) | 1.0 | 0.7 |
| TOTAL | 100.0 | 70.0 |

The composition described in Table 23 was used as a control composition which contains no silicon dioxide and 1% by weight of psilocybin.

TABLE 23
Unit Formula for Starcap Control Composition (1% Psilocybin).

| Excipient | Amount (%) | Mass (g) |
|---|---|---|
| Psilocybin | 1.0 | 0.7 |
| Pregelatinised Starch (StarCap) | 98.0 | 68.6 |
| Sodium Stearyl Fumarate (Pruv) | 1.0 | 0.7 |
| TOTAL | 100.0 | 70.0 |

The composition described in Table 24 was used to replicate the development formulation used in the previous 1% by weight of psilocybin tablet.

TABLE 24
Unit Formula for 1% psilocybin tablet composition.

| Excipient | Amount (%) | Mass (g) |
|---|---|---|
| Psilocybin | 1.0 | 0.7 |
| Silicified microcrystalline | 20.5 | 14.4 |
| cellulose (SMCC 50) | | |
| Silicified microcrystalline | 75.5 | 52.8 |
| cellulose (SMCC 90) | | |
| Sodium starch glycolate | 1.0 | 0.7 |
| Colloidal silicon dioxide (Aerosil) | 1.0 | 0.7 |
| Sodium stearyl fumarate (Pruv) | 1.0 | 0.7 |
| TOTAL | 100.0 | 70.0 |

The composition in Table 25 was used to replicate the development formulation used in the previous 5% by weight of psilocybin tablet.

TABLE 25
Unit Formula for 5% psilocybin tablet composition.

| Excipient | Amount (%) | Mass (g) |
|---|---|---|
| Psilocybin | 5.0 | 3.5 |
| Silicified Microcrystalline | 12.5 | 8.7 |
| Cellulose (SMCC 50) | | |
| Silicified Microcrystalline | 79.5 | 55.7 |
| Cellulose (SMCC 90) | | |
| Sodium Starch Glycolate | 1.0 | 0.7 |
| Colloidal silicon Dioxide (Aerosil) | 1.0 | 0.7 |
| Sodium Stearyl Fumarate (Pruv) | 1.0 | 0.7 |
| TOTAL | 100.0 | 70.0 |

Method: Each of the compositions described above were split equally into 6 separate HDPE sample bottles. Three of these bottles contained a 1 g desiccant sachet. All bottles were sealed shut with lids and parafilm. The bottles were subjected to the conditions as described in Table 26.

TABLE 26
Stability Storage Conditions

| Stability | | | | T = 1 | T = 3 | T = 6 |
|---|---|---|---|---|---|---|
| Conditions | Desiccant | T = 0* | T = 2 weeks | month | months | months |
| 25° C/60% RH | yes | Y | Y Assay/RS | Y | Y | Y |
| | | Assay/RS | | Assay/RS | Assay/RS | Assay/RS |
| 40° C/75% RH | yes | Y | Y Assay/RS | Y | Y | Y |
| | | Assay/RS | | Assay/RS | Assay/RS | Assay/RS |
| 60° C | yes | Y | Y Assay/RS | Y | Y | Y |
| | | Assay/RS | | Assay/RS | Assay/RS | Assay/RS |
| 25° C/60% RH | No | Y | Y Assay/RS | Y | Y | Y |
| | | Assay/RS | | Assay/RS | Assay/RS | Assay/RS |
| 40° C/75% RH | No | Y | Y Assay/RS | Y | Y | Y |
| | | Assay/RS | | Assay/RS | Assay/RS | Assay/RS |
| 60° C | No | Y | Y Assay/RS | Y | Y | Y |
| | | Assay/RS | | Assay/RS | Assay/RS | Assay/RS |

*T = 2 weeks and T = 1 month analysis was grouped together

The compositions were tested at for moisture content by Karl Fischer KF analysis and analysed by high performance liquid chromatography (HPLC) using the conditions described in Table 27. Analysis was performed at the start of the study (initial), 2 weeks, 1 month, 2 months and 6 months.

TABLE 27
HPLC parameters for testing compositions

| Column: | Waters XBridge C18, | | |
|---|---|---|---|
| | 3.5 | μm, | |
| | 150 × 4.6 | mm | |
| Column Oven: | 30° | C. | |
| Injection Volume | 20 | μL | |
| Autosampler Temperature: | Ambient or 20° | C. | |
| Flow Rate: | 1.0 | mL/min | |
| Detection: | UV 267 | nm | |
| Mobile Phase A | 95:5:0.1 | Water:MeOH:TFA | |
| Mobile Phase B | 95:5:0.1 | MeOH:Water:TFA | |
| Gradient Program | Time (mins) | % A | % B |
| | 0 | 100 | 0 |
| | 2 | 100 | 0 |
| | 15 | 0 | 100 |
| | 20 | 0 | 100 |
| | 20.1 | 100 | 0 |
| | 27 | 100 | 0 |

| | | |
|---|---|---|
| Needle Wash: | 90:10 | Water:MeOH |
| Run Time: | 27 | minutes |

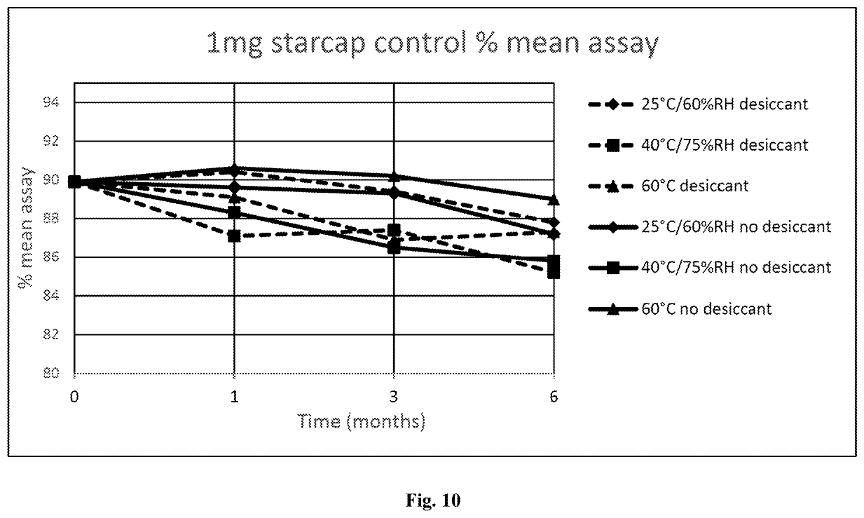

Table 28 and FIG. 10 show the results from storage of the Starcap control composition with and without desiccant at 25° C./60% relative humidity (RH), 40° C./75% RH, and 60° C. for two weeks, one month, three months and six months. The moisture content at T=0 of the composition was 7.11%, as determined by Karl Fisher analysis.

TABLE 28
Starcap Control Composition Analytical Testing Results

| Time | | Storage | Samples with desiccant | | | Samples without desiccant | | |
|---|---|---|---|---|---|---|---|---|
| Point | | Conditions | Assay | Related Substances | | Assay | Related Substances | |
| Initial | | N/A | Prep. 1 = 89.3% | Psilocin RRT | 1.52-0.5% | Prep. 1 = 89.3% | Psilocin RRT | 1.52-0.5% |
| | | | Prep. 2 = 90.5% | RRT | 1.36-0.1% | Prep. 2 = 90.5% | RRT | 1.36-0.1% |
| | | | Average = 89.9% | Total = 0.6% | | Average = 89.9% | Total = 0.6% | |
| 2 | week | 25° C./60% RH | Prep. 1 = 87.1% | Psilocin RRT | 1.58-0.5% | Prep. 1 = 88.1% | Psilocin RRT | 1.59-0.5% |
| | | | Prep. 2 = 87.2% | RRT | 1.41-0.1% | Prep. 2 = 88.1% | RRT | 1.42-0.1% |
| | | | Average = 87.2% | Total = 0.6% | | Average = 88.1% | Total = 0.5% | |
| | | 40° C./75% RH | Prep. 1 = 87.5% | Psilocin RRT | 1.59-0.5% | Prep. 1 = 89.2% | Psilocin RRT | 1.59-0.5% |
| | | | Prep. 2 = 87.1% | RRT | 1.42-0.1% | Prep. 2 = 88.7% | RRT | 1.42-0.1% |
| | | | Average = 87.3% | Total = 0.6% | | Average = 88.9% | Total = 0.6% | |
| | | 60° C. | Prep. 1 = 86.0% | Psilocin RRT | 1.60-0.5% | Prep. 1 = 89.6% | Psilocin RRT | 1.59-0.5% |
| | | | Prep. 2 = 84.2% | RRT | 1.42-0.1% | Prep. 2 = 88.5% | RRT | 1.42-0.1% |
| | | | Average = 85.1% | Total = 0.6% | | Average = 89.0% | Total = 0.6% | |
| 1 | month | 25° C./60% RH | Prep. 1 = 90.0% | Psilocin RRT | 1.58-0.5% | Prep. 1 = 89.9% | Psilocin RRT | 1.58-0.5% |
| | | | Prep. 2 = 90.9% | RRT | 1.41-0.1% | Prep. 2 = 89.3% | RRT | 1.41-0.1% |
| | | | Average = 90.4% | Total = 0.6% | | Average = 89.6% | Total = 0.6% | |
| | | 40° C./75% RH | Prep. 1 = 86.7% | Psilocin RRT | 1.58-0.5% | Prep. 1 = 88.1% | Psilocin RRT | 1.58-0.5% |
| | | | Prep. 2 = 87.6% | RRT | 1.41-0.1% | Prep. 2 = 88.4% | RRT | 1.41-0.1% |
| | | | Average = 87.1% | Total = 0.6% | | Average = 88.3% | Total = 0.6% | |
| | | 60° C. | Prep. 1 = 89.1% | Psilocin RRT | 1.58-0.6% | Prep. 1 = 90.8% | Psilocin RRT | 1.58-0.5% |
| | | | Prep. 2 = 89.0% | RRT | 1.42-0.1% | Prep. 2 = 90.4% | RRT | 1.41-0.1% |
| | | | Average = 89.1% | Total = 0.6% | | Average = 90.6% | Total = 0.6% | |
| 3 | months | 25° C./60% RH | Prep. 1 = 89.4% | Psilocin RRT | 1.51-0.5% | Prep. 1 = 89.3% | Psilocin RRT | 1.51-0.5% |
| | | | Prep. 2 = 89.5% | RRT | 1.36-0.1% | Prep. 2 = 89.2% | RRT | 1.36-0.1% |
| | | | Average = 89.4% | Total = 0.6% | | Average = 89.3% | Total = 0.6% | |
| | | 40° C./75% RH | Prep. 1 = 87.3% | Psilocin RRT | 1.51-0.5% | Prep. 1 = 86.5% | Psilocin RRT | 1.51-0.4% |
| | | | Prep. 2 = 87.4% | RRT | 1.36-0.1% | Prep. 2 = 86.6% | RRT | 1.36-0.1% |
| | | | Average = 87.4% | Total = 0.6% | | Average = 86.5% | Total = 0.5% | |
| | | 60° C. | Prep. 1 = 86.3% | Psilocin RRT | 1.65-0.6% | Prep. 1 = 90.7% | Psilocin RRT | 1.65-0.5% |
| | | | Prep. 2 = 87.5% | RRT | 1.50-0.1% | Prep. 2 = 89.7% | RRT | 1.51-0.1% |
| | | | Average = 86.9% | Total = 0.7% | | Average = 90.2% | Total = 0.6% | |
| 6 | months | 25° C./60% RH | Prep. 1 = 87.8% | Psilocin RRT | 1.57-0.55% | Prep. 1 = 86.9% | Psilocin RRT | 1.57-0.56% |
| | | | Prep. 2 = 87.8% | RRT | 1.41-0.06% | Prep. 2 = 87.6% | RRT | 1.41-0.06% |
| | | | Average = 87.8% | Total = 0.6% | | Average = 87.2% | Total = 0.6% | |
| | | 40° C./75% RH | Prep. 1 = 85.4% | Psilocin RRT | 1.56-0.40% | Prep. 1 = 86.2% | Psilocin RRT | 1.56-0.40% |
| | | | Prep. 2 = 85.1% | RRT | 1.40-0.07% | Prep. 2 = 85.4% | RRT | 1.40-0.06% |
| | | | Average = 85.2% | Total = 0.5% | | Average = 85.8% | Total = 0.5% | |
| | | 60° C. | Prep. 1 = 87.2% | Psilocin RRT | 1.57-0.66% | Prep. 1 = 90.3% | Psilocin RRT | 1.57-0.58% |
| | | | Prep. 2 = 87.4% | RRT | 1.41-0.09% | Prep. 2 = 87.6% | RRT | 1.40-0.10% |

| | | | |
|---|---|---|---|
| Average = 87.3% | Total = 0.8% | Average = 89.0% | Total = 0.7% |

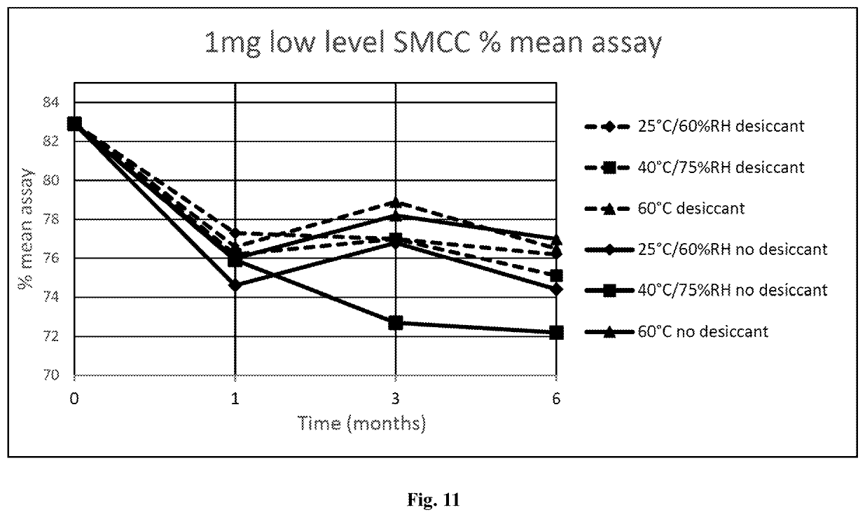

Table 29 and FIG. 11 show the results from storage of the 1 mg psilocybin low level SMCC composition with and without desiccant at 25° C./60% RH, 40° C./75% RH, and 60° C. for two weeks, one month, three months and six months. The moisture content at T=0 of the composition was 6.77%, as determined by Karl Fisher analysis.

TABLE 29
1 mg Low Level SMCC Composition Analytical Testing Results

| Time | | Storage | Samples with desiccant | | | Samples without desiccant | | |
|---|---|---|---|---|---|---|---|---|
| Point | | Conditions | Assay | Related Substances | | Assay | Related Substances | |
| Initial | | N/A | Prep. 1 = 84.0% | Psilocin RRT | 1.51-0.4% | Prep. 1 = 84.0% | Psilocin RRT | 1.51-0.4% |
| | | | Prep. 2 = 81.9% | RRT | 1.36-0.1% | Prep. 2 = 81.9% | RRT | 1.36-0.1% |
| | | | Average = 82.9% | Total = 0.5% | | Average = 82.9% | Total = 0.5% | |
| 2 | week | 25° C./60% RH | Prep. 1 = 66.6% | Psilocin RRT | 1.60-0.4% | Prep. 1 = 67.4% | Psilocin RRT | 1.59-0.4% |
| | | | Prep. 2 = 67.6% | RRT | 1.42-0.1% | Prep. 2 = 69.5% | RRT | 1.42-0.1% |
| | | | Average = 67.1% | Total = 0.4% | | Average = 68.5% | Total = 0.5% | |
| | | 40° C./75% RH | Prep. 1 = 66.8% | Psilocin RRT | 1.60-0.4% | Prep. 1 = 69.8% | Psilocin RRT | 1.59-0.4% |
| | | | Prep. 2 = 68.4% | RRT | 1.42-0.1% | Prep. 2 = 68.6% | RRT | 1.42-0.1% |
| | | | Average = 67.6% | Total = 0.4% | | Average = 69.2% | Total = 0.4% | |
| | | 60° C. | Prep. 1 = 70.1% | Psilocin RRT | 1.59-0.4% | Prep. 1 = 68.8% | Psilocin RRT | 1.59-0.4% |
| | | | Prep. 2 = 70.6% | RRT | 1.42-0.1% | Prep. 2 = 71.2% | RRT | 1.42-0.1% |
| | | | Average = 70.3% | Total = 0.5% | | Average = 70.0% | Total = 0.4% | |
| 1 | month | 25° C./60% RH | Prep. 1 = 77.7% | Psilocin RRT | 1.59-0.4% | Prep. 1 = 76.6% | Psilocin RRT | 1.59-0.4% |
| | | | Prep. 2 = 77.0% | RRT | 1.42-0.1% | Prep. 2 = 72.6% | RRT | 1.42-0.1% |
| | | | Average = 77.3% | Total = 0.5% | | Average = 74.6% | Total = 0.4% | |
| | | 40° C./75% RH | Prep. 1 = 76.8% | Psilocin RRT | 1.59-0.4% | Prep. 1 = 76.2% | Psilocin RRT | 1.59-0.3% |
| | | | Prep. 2 = 75.6% | RRT | 1.42-0.1% | Prep. 2 = 75.6% | RRT | 1.42-0.1% |
| | | | Average = 76.2% | Total = 0.4% | | Average = 75.9% | Total = 0.4% | |
| | | 60° C. | Prep. 1 = 77.6% | Psilocin RRT | 1.59-0.4% | Prep. 1 = 75.8% | Psilocin RRT | 1.59-0.4% |
| | | | Prep. 2 = 75.5% | RRT | 1.42-0.1% | Prep. 2 = 76.2% | RRT | 1.42-0.1% |
| | | | Average = 76.6% | Total = 0.5% | | Average = 76.0% | Total = 0.4% | |
| 3 | months | 25° C./60% RH | Prep. 1 = 76.1% | Psilocin RRT | 1.51-0.4% | Prep. 1 = 75.7% | Psilocin RRT | 1.51-0.4% |
| | | | Prep. 2 = 77.8% | RRT | 1.36-0.1% | Prep. 2 = 77.9% | RRT | 1.36-0.1% |
| | | | Average = 77.0% | Total = 0.5% | | Average = 76.8% | Total = 0.5% | |
| | | 40° C./75% RH | Prep. 1 = 76.8% | Psilocin RRT | 1.51-0.3% | Prep. 1 = 73.1% | Psilocin RRT | 1.51-0.2% |
| | | | Prep. 2 = 77.2% | RRT | 1.35-0.1% | Prep. 2 = 72.3% | RRT | 1.35-0.1% |
| | | | Average = 77.0% | Total = 0.4% | | Average = 72.7% | Total = 0.3% | |
| | | 60° C. | Prep. 1 = 79.6% | Psilocin RRT | 1.65-0.4% | Prep. 1 = 80.7% | Psilocin RRT | 1.65-0.3% |
| | | | Prep. 2 = 78.2% | RRT | 1.50-0.1% | Prep. 2 = 75.7% | RRT | 1.50-0.1% |
| | | | Average = 78.9% | RRT | 1.62-0.1% | Average = 78.2% | RRT | 1.62-0.1% |
| | | | | Total = 0.5% | | | Total = 0.5% | |
| 6 | months | 25° C./60% RH | Prep. 1 = 78.1% | Psilocin RRT | 1.56-0.45% | Prep. 1 = 72.6% | Psilocin RRT | 1.56-0.44% |
| | | | Prep. 2 = 74.4% | RRT | 1.41-0.06% | Prep. 2 = 76.2% | RRT | 1.41-0.06% |
| | | | Average = 76.2% | Total = 0.5% | | Average = 74.4% | Total = 0.5% | |
| | | 40° C./75% RH | Prep. 1 = 74.2% | Psilocin RRT | 1.56-0.23% | Prep. 1 = 75.2% | Psilocin RRT | 1.56-0.19% |
| | | | Prep. 2 = 76.0% | RRT | 1.40-0.07% | Prep. 2 = 69.3% | RRT | 1.40-0.06% |
| | | | Average = 75.1% | Total = 0.3% | | Average = 72.2% | Total = 0.3% | |
| | | 60° C. | Prep. 1 = 75.7% | Psilocin RRT | 1.56-0.48% | Prep. 1 = 76.3% | Psilocin RRT | 1.57-0.40% |

| | | | | |
|---|---|---|---|---|
| Prep. 2 = 77.3% | RRT 1.41-0.10% | Prep. 2 = 77.7% | RRT | 1.41-0.10% |

| | | | |
|---|---|---|---|
| Average = 76.5% | Total = 0.6% | Average = 77.0% | Total = 0.5% |

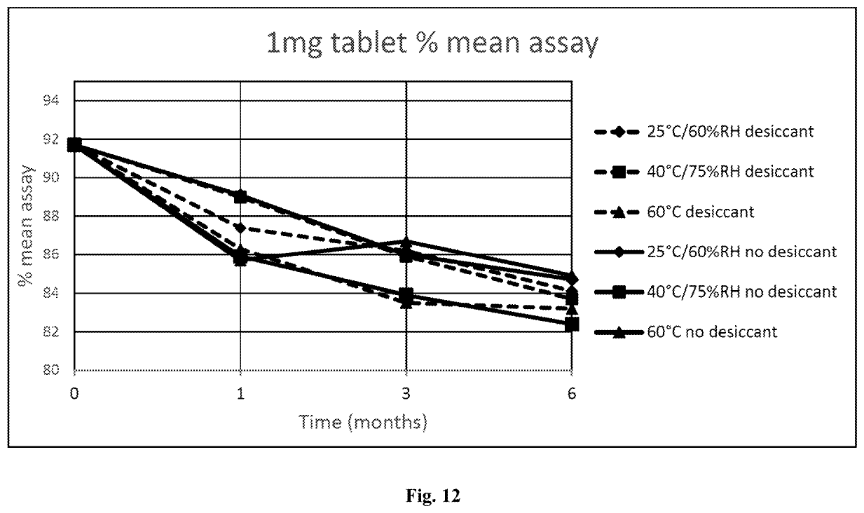

Table 30 and FIG. 12 show the results from storage of the 1 mg psilocybin tablet composition with and without desiccant at 25° C./60% RH, 40° C./75% RH, and 60° C. for two weeks, one month, three months and six months. The moisture content at T=0 of the composition was 3.05%, as determined by Karl Fisher analysis.

TABLE 30
1 mg Psilocybin Tablet Composition Analytical Testing Results

| Time | | Storage | Samples With desiccant | | | Samples Without desiccant | | |
|---|---|---|---|---|---|---|---|---|
| Point | | Conditions | Assay | Related Substances | | Assay | Related Substances | |
| Initial | | N/A | Prep. 1 = 93.0% | Psilocin RRT | 1.51-0.3% | Prep. 1 = 93.0% | Psilocin RRT | 1.51-0.3% |
| | | | Prep. 2 = 90.4% | RRT 1.36- | 0.1% | Prep. 2 = 90.4% | RRT | 1.36-0.1% |
| | | | Average = 91.7% | Total = 0.3% | | Average = 91.7% | Total = 0.3% | |
| 2 | week | 25° C./60% RH | Prep. 1 = 77.9% | Psilocin RRT | 1.59-0.3% | Prep. 1 = 83.2% | Psilocin RRT | 1.59-0.3% |
| | | | Prep. 2 = 77.0% | RRT | 1.42-0.1% | Prep. 2 = 84.3% | RRT | 1.42-0.1% |
| | | | Average = 77.4% | Total = 0.3% | | Average = 83.7% | Total = 0.3% | |
| | | 40° C./75% RH | Prep. 1 = 80.4% | Psilocin RRT | 1.59-0.3% | Prep. 1 = 84.6% | Psilocin RRT | 1.59-0.3% |
| | | | Prep. 2 = 81.1% | RRT | 1.42-0.1% | Prep. 2 = 85.3% | RRT | 1.42-0.1% |
| | | | Average = 80.7% | Total = 0.4% | | Average = 84.9% | Total = 0.3% | |
| | | 60° C. | Prep. 1 = 78.4% | Psilocin RRT | 1.59-0.3% | Prep. 1 = 83.5% | Psilocin RRT | 1.59-0.3% |
| | | | Prep. 2 = 81.1% | RRT | 1.42-0.1% | Prep. 2 = 83.2% | RRT | 1.42-0.1% |
| | | | Average = 79.7% | Total = 0.4% | | Average = 83.4% | Total = 0.4% | |
| 1 | month | 25° C./60% RH | Prep. 1 = 87.6% | Psilocin RRT | 1.59-0.3% | Prep. 1 = 89.7% | Psilocin RRT | 1.59-0.3% |
| | | | Prep. 2 = 87.1% | RRT | 1.42-0.1% | Prep. 2 = 88.5% | RRT | 1.42-0.1% |
| | | | Average = 87.4% | Total = 0.4% | | Average = 89.1% | Total = 0.3% | |
| | | 40° C./75% RH | Prep. 1 = 89.3% | Psilocin RRT | 1.59-0.3% | Prep. 1 = 88.1% | Psilocin RRT | 1.59-0.2% |
| | | | Prep. 2 = 88.7% | RRT | 1.42-0.1% | Prep. 2 = 83.6% | RRT | 1.42-0.1% |
| | | | Average = 89.0% | Total = 0.3% | | Average = 85.9% | Total = 0.3% | |
| | | 60° C. | Prep. 1 = 86.1% | Psilocin RRT | 1.59-0.3% | Prep. 1 = 85.0% | Psilocin RRT | 1.59-0.3% |
| | | | Prep. 2 = 86.4% | RRT | 1.42-0.1% | Prep. 2 = 86.4% | RRT | 1.42-0.1% |
| | | | Average = 86.3% | Total = 0.4% | | Average = 85.7% | Total = 0.4% | |
| 3 | months | 25° C./60% RH | Prep. 1 = 85.6% | Psilocin RRT | 1.51-0.3% | Prep. 1 = 85.0% | Psilocin RRT | 1.51-0.3% |
| | | | Prep. 2 = 86.7% | RRT | 1.36-0.1% | Prep. 2 = 86.9% | RRT | 1.36-0.1% |
| | | | Average = 86.2% | Total = 0.3% | | Average = 86.0% | Total = 0.3% | |
| | | 40° C./75% RH | Prep. 1 = 85.9% | Psilocin RRT | 1.51-0.2% | Prep. 1 = 83.2% | Psilocin RRT | 1.51-0.2% |
| | | | Prep. 2 = 85.8% | RRT | 0.70-0.1% | Prep. 2 = 84.6% | RRT | 0.70-0.2% |
| | | | Average = 85.9% | RRT | 1.35-0.1% | Average = 83.9% | RRT | 1.35-0.1% |
| | | | | Total = 0.4% | | | Total = 0.4% | |
| | | 60° C. | Prep. 1 = 83.8% | Psilocin RRT | 1.64-0.3% | Prep. 1 = 86.1% | Psilocin RRT | 1.64-0.3% |
| | | | Prep. 2 = 83.3% | RRT | 1.50-0.1% | Prep. 2 = 87.4% | RRT | 1.44-0.1% |
| | | | Average = 83.5% | Total = 0.4% | | Average = 86.7% | RRT | 1.50-0.1% |
| | | | | | | | Total = 0.5% | |
| 6 | months | 25° C./60% RH | Prep. 1 =84.3% | Psilocin RRT | 1.56-0.29% | Prep. 1 = 85.3% | Psilocin RRT | 1.56-0.29% |
| | | | Prep. 2 = 83.9% | RRT | 1.40-0.07% | Prep. 2 = 84.1% | RRT | 1.41-0.1% |
| | | | Average = 84.1% | Total = 0.4% | | Average = 84.7% | Total = 0.5% | |
| | | 40° C./75% RH | Prep. 1 = 83.6% | Psilocin RRT 1 | .56-0.12% | Prep. 1 = 82.7% | Psilocin RRT | 1.56-0.13% |
| | | | Prep. 2 = 83.8% | RRT | 1.40-0.09% | Prep. 2 = 82.1% | RRT | 1.40-0.08% |
| | | | Average = 83.7% | Total = 0.2% | | Average = 82.4% | Total = 0.2% | |
| | | 60° C. | Prep. 1 = 82.6% | Psilocin RRT | 1.57-0.34% | Prep. 1 = 84.6% | Psilocin RRT | 1.56-0.36% |
| | | | Prep. 2 = 83.8% | RRT | 1.40-0.11% | Prep. 2 = 85.2% | RRT | 1.40-0.13% |

| | | | |
|---|---|---|---|
| Average = 83.2% | Total = 0.5% | Average = 84.9% | Total = 0.5% |

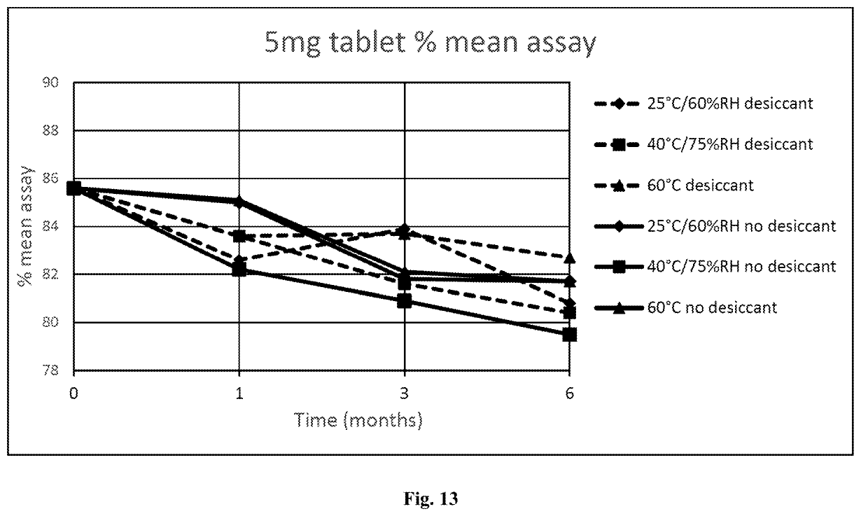

Table 31 and FIG. 13 show the results from storage of the 5% psilocybin tablet composition with and without desiccant at 25° C./60% RH, 40° C./75% RH, and 60° C. for two weeks, one month, three months and six months. The moisture content at T=0 of the composition was 2.72%, as determined by Karl Fisher analysis.

TABLE 31
5 mg Psilocybin Tablet Composition Analytical Testing Results

| Time | | Storage | Samples With desiccant | | | Samples Without desiccant | | |
|---|---|---|---|---|---|---|---|---|
| Point | | Conditions | Assay | Related Substances | | Assay | Related Substances | |
| Initial | | N/A | Prep. 1 = 86.5% | Psilocin RRT | 1.51-0.4% | Prep. 1 = 86.5% | Psilocin RRT | 1.51-0.4% |
| | | | Prep. 2 = 84.7% | RRT | 1.36-0.1% | Prep. 2 = 84.7% | RRT | 1.36-0.1% |
| | | | Average = 85.6% | Total = 0.5% | | Average = 85.6% | Total = 0.5% | |
| 2 | week | 25° C./60% RH | Prep. 1 = 78.4% | Psilocin RRT | 1.59-0.4% | Prep. 1 = 81.2% | Psilocin RRT | 1.59-0.4% |
| | | | Prep. 2 = 77.8% | RRT | 1.42-0.1% | Prep. 2 = 81.0% | RRT | 1.42-0.1% |
| | | | Average = 78.1% | Total = 0.4% | | Average = 81.1% | Total = 0.5% | |
| | | 40° C./75% RH | Prep. 1 = 76.8% | Psilocin RRT | 1.59-0.4% | Prep. 1 = 76.9% | Psilocin RRT | 1.59-0.4% |
| | | | Prep. 2 = 75.1% | RRT | 1.42-0.1% | Prep. 2 = 79.8% | RRT | 1.42-0.1% |
| | | | Average = 76.0% | Total = 0.5% | | Average = 78.4% | Total = 0.5% | |
| | | 60° C. | Prep. 1 = 80.6% | Psilocin RRT | 1.59-0.4% | Prep. 1 = 83.6% | Psilocin RRT | 1.59-0.4% |
| | | | Prep. 2 = 81.0% | RRT | 1.42-0.1% | Prep. 2 = 79.9% | RRT | 1.42-0.1% |
| | | | Average = 80.8% | Total = 0.5% | | Average = 81.7% | Total = 0.5% | |
| 1 | month | 25° C./60% RH | Prep. 1 = 82.9% | Psilocin RRT | 1.59-0.4% | Prep. 1 = 86.0% | Psilocin RRT | 1.59-0.4% |
| | | | Prep. 2 = 82.3% | RRT | 1.42-0.1% | Prep. 2 = 84.0% | RRT | 1.42-0.1% |
| | | | Average = 82.6% | Total = 0.5% | | Average = 85.0% | Total = 0.5% | |
| | | 40° C./75% RH | Prep. 1 = 83.9% | Psilocin RRT | 1.59-0.4% | Prep. 1 = 82.8% | Psilocin RRT | 1.59-0.4% |
| | | | Prep. 2 = 83.4% | RRT | 1.42-0.1% | Prep. 2 = 81.6% | RRT | 1.42-0.1% |
| | | | Average = 83.6% | Total = 0.5% | | Average = 82.2% | Total = 0.4% | |
| | | 60° C. | Prep. 1 = 83.4% | Psilocin RRT | 1.59-0.4% | Prep. 1 = 83.8% | Psilocin RRT | 1.59-0.4% |
| | | | Prep. 2 = 83.9% | RRT | 1.42-0.1% | Prep. 2 = 86.4% | RRT | 1.42-0.1% |
| | | | Average = 83.6% | Total = 0.5% | | Average = 85.1% | Total = 0.5% | |
| 3 | months | 25° C./60% RH | Prep. 1 = 84.2% | Psilocin RRT | 1.51-0.4% | Prep. 1 = 82.3% | Psilocin RRT | 1.51-0.4% |
| | | | Prep. 2 = 83.6% | RRT | 1.36-0.1% | Prep. 2 = 81.3% | RRT | 1.36-0.1% |
| | | | Average = 83.9% | Total = 0.5% | | Average = 81.8% | Total = 0.5% | |
| | | 40° C./75% RH | Prep. 1 = 81.7% | Psilocin RRT | 1.50-0.4% | Prep. 1 = 80.3% | Psilocin RRT | 1.51-0.3% |
| | | | Prep. 2 = 81.6% | RRT | 1.35-0.1% | Prep. 2 = 81.5% | RRT | 1.35-0.1% |
| | | | Average = 81.6% | Total = 0.4% | | Average = 80.9% | Total = 0.4% | |
| | | 60° C. | Prep. 1 = 83.4% | Psilocin RRT | 1.64-0.5% | Prep. 1 = 82.1% | Psilocin RRT | 1.64-0.5% |
| | | | Prep. 2 = 83.9% | RRT | 1.49-0.1% | Prep. 2 = 82.1% | RRT | 1.49-0.1% |
| | | | Average = 83.7% | Total = 0.6% | | Average = 82.1% | Total = 0.6% | |
| 6 | months | 25° C./60% RH | Prep. 1 = 81.3% | Psilocin RRT | 1.56-0.47% | Prep. 1 = 82.1% | Psilocin RRT | 1.56- 0.46% |
| | | | Prep. 2 = 80.2% | RRT | 1.40-0.06% | Prep. 2 = 81.3% | RRT | 1.40-0.07% |
| | | | Average = 80.8% | Total = 0.5% | | Average = 81.7% | Total = 0.5% | |
| | | 40° C./75% RH | Prep. 1 =8 0.2% | Psilocin RRT | 1.56-0.31% | Prep. 1 = 79.9% | Psilocin RRT | 1.56-0.29% |
| | | | Prep. 2 = 80.5% | RRT | 1.40-0.08% | Prep. 2 = 79.2% | RRT | 1.40-0.08% |
| | | | Average = 80.4% | Total = 0.4% | | Average = 79.5% | Total = 0.4% | |
| | | 60° C. | Prep. 1 = 83.0% | Psilocin RRT | 1.57-0.60% | Prep. 1 = 81.5% | Psilocin RRT | 1.57-0.57% |
| | | | Prep. 2 = 82.4% | RRT | 1.41-0.10% | Prep. 2 = 81.9% | RRT | 1.41-0.11% |

| | | | |
|---|---|---|---|
| Average = 82.7% | Total = 0.7% | Average = 81.7% | Total = 0.7% |

Table 32 shows the assay results for the 1 mg psilocybin capsule control sample stability study.

TABLE 32
1 mg Psilocybin Capsule Control Sample Assay Results

| Timepoint | Mean % Assay |
|---|---|
| T = 2 weeks & 1 month | 101.5 |
| T = 3 months | 97.1 |
| T = 6 months | 100.6 |

Results: For all studies the level of Psilocin impurity remained unchanged throughout the time of the study.

The compositions containing silicon dioxide excipients showed an increasing downward trend in the assay when compared to the control composition (see Table 33). In addition, Table 33 indicates that the use of a desiccant has a limited effect on composition stability.

TABLE 33
Differences in % Assay from T-0 months to T = 6 months

| | 1% psilocybin Starcap | | | | | | | |
|---|---|---|---|---|---|---|---|---|
| | control | | 1% low level SMCC | | 1% psilocybin tablet | | 5% psilocybin tablet | |
| Storage | | no | | no | | no | | no |
| Condition | desiccant | desiccant | desiccant | desiccant | desiccant | desiccant | desiccant | desiccant |
| 25° C/60% RH | −2.1 | −2.7 | −6.7 | −8.5 | −7.6 | −7.0 | −4.8 | −3.9 |
| 40° C/75% RH | −4.7 | −4.1 | −7.8 | −10.7 | −8.0 | −7.3 | −5.2 | −6.1 |
| 60° C | −2.6 | −0.9 | −6.4 | −5.9 | −8.5 | −6.8 | −2.9 | −3.9 |

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

### EMBODIMENTS

1. A pharmaceutical composition comprising:
a therapeutically effective amount of psilocybin; and
one or more pharmaceutically acceptable excipients,
   wherein, after storage of the composition at 40° C. and 75% relative humidity for one month, the potency of the psilocybin in the composition decreases by less than 5% and the mass balance of psilocybin and related substances is greater than 97%.

2. The pharmaceutical composition of embodiment 1, wherein the composition comprises at least one pharmaceutically acceptable excipient that does not substantially chemically or physically interact with the phosphonate ester of psilocybin.

3. The pharmaceutical composition of any one of embodiments 1-2, wherein the composition does not contain a pharmaceutically acceptable excipient that substantially chemically or physically interacts with the phosphonate ester of psilocybin.

4. The pharmaceutical composition of any one of embodiments 1-3, wherein the composition does not contain an inorganic silicate, silicon dioxide or a silicified excipient.

5. The pharmaceutical composition of any one of embodiments 1-4, wherein the composition does not contain aluminum calcium silicate, calcium silicate, diatomaceous earth, magnesium silicate, Perlite, potassium silicate, silica aerogel, silicon dioxide, sodium aluminosilicate, sodium calcium aluminosilicate, sodium silicate, talc (basic magnesium silicate) or tricalcium silicate.

6. The pharmaceutical composition of any one of embodiments 1-5, wherein the composition does not contain silicified microcrystalline cellulose.

7. The pharmaceutical composition of any one of embodiments 1-6, wherein the composition does not contain polyols, mannitol, xylitol, sucrose, sorbitol or fructose.

8. The pharmaceutical composition of any one of embodiments 1-7, wherein the composition does not contain calcium carbonate or calcium phosphate.

9. The pharmaceutical composition of any one of embodiments 1-8, wherein the composition does not contain a glidant.

10. The pharmaceutical composition of any one of embodiments 1-9, wherein the one or more pharmaceutically acceptable excipients comprises a diluent.

11. The pharmaceutical composition of any one of embodiments 1-10, wherein the one or more pharmaceutically acceptable excipients comprises a lubricant.

12. The pharmaceutical composition of any one of embodiments 1-11, wherein the composition comprises:
a) about 85-99% by weight of a diluent; and
b) about 0.5-2.0% by weight of a lubricant.

13. The pharmaceutical composition of any one of embodiments 1-12, wherein the composition comprises about 1%-10% by weight of psilocybin.

14. The pharmaceutical composition of any one of embodiments 10-13, wherein the diluent is pregelatinized starch.

15. The pharmaceutical composition of any one of embodiments 11-14, wherein the lubricant is selected from the group consisting of sodium stearyl fumarate, magnesium stearate, calcium stearate, zinc stearate, glyceryl monostearate, glyceryl behenate, glyceryl palmostearate, magnesium lauryl stearate, stearic acid, talc, and hydrogenated castor oil.

16. The pharmaceutical composition of embodiment 15, wherein the lubricant is sodium stearyl fumarate.

17. The pharmaceutical composition of any one of embodiments 1-16, wherein the chemical purity of psilocybin in the composition is not less than 97% as determined by HPLC analysis.

18. The pharmaceutical composition of any one of embodiments 1-17, wherein the composition comprises 1 mg to 40 mg of psilocybin.

19. The pharmaceutical composition of any one of embodiments 1-17, wherein the composition comprises 1 mg of psilocybin

20. The pharmaceutical composition of any one of embodiments 1-17, wherein the composition comprises 5 mg of psilocybin.

21. The pharmaceutical composition of any one of embodiments 1-17, wherein the composition comprises 10 mg of psilocybin.

22. The pharmaceutical composition of any one of embodiments 1-17, wherein the composition comprises 25 mg of psilocybin.

23. The pharmaceutical composition of any one of embodiments 1-22, wherein the psilocybin comprises a crystalline psilocybin.

24. The pharmaceutical composition of embodiment 23, wherein the crystalline psilocybin is crystalline Polymorph A.

25. The pharmaceutical composition of embodiment 23, wherein the crystalline psilocybin is crystalline Hydrate A.

26. The pharmaceutical composition of embodiment 23, wherein the crystalline psilocybin is crystalline Polymorph A′.

27. The pharmaceutical composition of embodiment 23, wherein the crystalline psilocybin is crystalline Polymorph B.

28. The pharmaceutical composition of any one of embodiments 1-22, wherein the psilocybin is amorphous.

29. The pharmaceutical composition of any one of embodiments 1-28, wherein the composition is a capsule.

30. The pharmaceutical composition of any one of embodiments 1-28, wherein the composition is a tablet.

31. The pharmaceutical composition of any one of embodiments 1-22, wherein the composition is a solution.

32. The pharmaceutical composition of any one of embodiments 1-30, wherein the content uniformity of the composition complies with the European Pharmacopeia 2.96, or USP <905>.

33. A method of making a pharmaceutical composition comprising:
a) direct mixing psilocybin and one or more pharmaceutically acceptable excipients to provide a blend, and
b) filling a capsule with the blend to provide the pharmaceutical composition,
wherein the content uniformity of the composition complies with the European Pharmacopeia 2.96, or USP <905>.

34. The method of embodiment 33, wherein the mass balance of psilocybin and related substances in the composition decreases by less than 3% after storage of the composition at 40° C. and 75% relative humidity for one month.

35. The method of any one of embodiments 33-34, wherein the blend comprises about 1% to about 10% by weight of psilocybin.

36. The method of any one of embodiments 33-35, wherein the capsule contains 1 mg, 5 mg, 10 mg or 25 mg of psilocybin.

37. The method of any one of embodiments 33-36, wherein the one or more pharmaceutically acceptable excipients comprises a diluent.

38. The method of any one of embodiments 33-37, wherein the blend further comprises a lubricant.

39. The method of any one of embodiments 33-38, wherein the blend does not contain a glidant.

40. A pharmaceutical composition prepared by the process of any one of embodiments 33-39.

41. A method of treating a mood disorder, comprising administering a therapeutically effective amount of the composition of any one of embodiments 1-32 and 40.

1. A pharmaceutical composition comprising:
about 1-25 mg of psilocybin; and
about 85% to about 99%, by weight, partially pregelatinized starch.

2. The pharmaceutical composition of claim 1, further comprising a lubricant.

3. The pharmaceutical composition of claim 2, wherein the composition comprises about 0.5% to about 2.0%, by weight of the lubricant.

4. The pharmaceutical composition of claim 3, wherein the lubricant is sodium stearyl fumarate.

5. The pharmaceutical composition of claim 1, wherein the composition is a capsule.

6. The pharmaceutical composition of claim 1, wherein the composition comprises about 1% to about 10%, by weight, psilocybin.

7. The pharmaceutical composition of claim 1, wherein the composition does not contain silicified microcrystalline cellulose.

8. The pharmaceutical composition of claim 1, wherein the composition comprises 1 mg of psilocybin.

9. The pharmaceutical composition of claim 1, wherein the composition comprises 5 mg of psilocybin.

10. The pharmaceutical composition of claim 1, wherein the composition comprises 10 mg of psilocybin.

11. The pharmaceutical composition of claim 1, wherein the composition comprises 25 mg of psilocybin.

12. A pharmaceutical composition comprising:
about 1% to about 10%, by weight, psilocybin;
about 85% to about 99%, by weight, partially pregelatinized starch; and
about 0.5% to about 2.0%, by weight, sodium stearyl fumarate.

13. The pharmaceutical composition of claim 12, wherein the composition does not contain silicified microcrystalline cellulose.

14. The pharmaceutical composition of claim 12, wherein the composition is a capsule.

15. The pharmaceutical composition of claim 12, wherein the composition comprises 1 mg of psilocybin.

16. The pharmaceutical composition of claim 12, wherein the composition comprises 5 mg of psilocybin.

17. The pharmaceutical composition of claim 12, wherein the composition comprises 10 mg of psilocybin.

18. The pharmaceutical composition of claim 12, wherein the composition comprises 25 mg of psilocybin.

19. The pharmaceutical composition of claim 12, wherein the composition comprises 40 mg of psilocybin.

20. The pharmaceutical composition of claim 1, wherein the content uniformity of the composition complies with the European Pharmacopeia 2.96, or USP <905>.

21. The pharmaceutical composition of claim 12, wherein the content uniformity of the composition complies with the European Pharmacopeia 2.96, or USP <905>.

22. A pharmaceutical composition comprising:
about 1% to about 10%, by weight, psilocybin;
about 85% to about 99%, by weight, diluent;
wherein the potency of the psilocybin in the composition decreases by less than 5%, and wherein the content uniformity of the composition complies with the European Pharmacopeia 2.96, or USP <905>.

23. The pharmaceutical composition of claim 22, wherein the diluent has an average particle size in the range of 70 microns to 120 microns.

24. The pharmaceutical composition of claim 22, wherein the diluent has an average particle size in the range of 70 microns to 100 microns.

25. The pharmaceutical composition of claim 22, wherein the diluent has a monomodal particle size distribution.

26. The pharmaceutical composition of claim 25, wherein the diluent has an average particle size in the range of 70 microns to 120 microns.

27. The pharmaceutical composition of claim 22, wherein the diluent comprises a single starch product.

28. The pharmaceutical composition of claim 22, wherein the diluent has an average particle size in the range of 70 microns to 120 microns.
